SEQUENCE LISTING (4 sequences)

SEQ ID NO: 1 - DNA, 11 bp, Homo sapiens
  misc_feature 1..11: note = oligo DNA derived from Exon 2 of WT KRAS gene sense chain
  source 1..11: mol_type = genomic DNA
ggtggcgtagg

SEQ ID NO: 2 - DNA, 11 bp, Homo sapiens
  misc_feature 1..11: note = oligo DNA derived from Exon 2 of WT KRAS gene antisense chain
  source 1..11: mol_type = genomic DNA
cctacgccacc

SEQ ID NO: 3 - DNA, 11 bp, synthetic construct
  misc_feature 1..11: note = oligo DNA derived from mutant KRAS gene sense chain
  source 1..11: mol_type = other DNA
ggtgacgtagg

SEQ ID NO: 4 - DNA, 11 bp, synthetic construct
  modified_base 1..2: mod_base = OTHER; note = 2',4'-BNANC(N-H)-cytosine
  modified_base 3: mod_base = OTHER; note = 2',4'-BNANC(N-H)- thymine
  modified_base 5: mod_base = OTHER; note = 2',4'-BNANC(N-H)-cytosine
  modified_base 7..8: mod_base = OTHER; note = 2',4'-BNANC(N-H)-cytosine
  modified_base 10..11: mod_base = OTHER; note = 2',4'-BNANC(N-H)-cytosine
  source 1..11: mol_type = other DNA
cctacgccacc

(19) United States
(12) Patent Application Publication
(10) Pub. No.: US 2025/0043328 A1
(43) Pub. Date: Feb. 6, 2025

(54) AGENT FOR RAISING MELTING TEMPERATURE OF DOUBLE-STRANDED NUCLEIC ACID, AND USES THEREFOR

(71) Applicant: NITTO BOSEKI CO., LTD., Fukushima-shi, Fukushima (JP)

(72) Inventors: Nahohiro FUJIMURA, Kawasaki (JP); Ami TACHIBANA, Kawasaki (JP); Tomoaki UCHIKI, Kawasaki (JP); Minoru TAKEUCHI, Koriyama (JP); Yoko TERUUCHI, Koriyama (JP); Koji WATANABE, Koriyama (JP)

(73) Assignee: NITTO BOSEKI CO., LTD., Fukushima-shi, Fukushima (JP)

(21) Appl. No.: 18/715,009

(22) PCT Filed: Dec. 1, 2022
(86) PCT No.: PCT/JP2022/044338

§ 371 Date: May 30, 2024

(30) Foreign Application Priority Data
Dec. 10, 2021 (JP) 2021-201162

(51) Int. Cl. C12Q 1/6806; C12Q 1/6816; C12Q 1/6827

(52) U.S. Cl. CPC C12Q 1/6806; C12Q 1/6816; C12Q 1/6827

(57) ABSTRACT

In order to address problems in the prior art, the present invention provides a novel agent for raising the melting temperature (Tm) of a double-stranded nucleic acid, methods for detecting mismatching of a double-stranded nucleic acid or mutation with respect to a standard nucleic acid in a sample nucleic acid, and kits for carrying out these methods.

Embodiments of the present invention include: an agent for raising the melting temperature of a double-stranded nucleic acid, said agent containing an amphoteric copolymer that includes a specific cationic constituent unit (1) and a specific anionic constituent unit (2); a method for detecting a nucleic acid mismatch and a method for detecting mutation with respect to a standard nucleic acid in a sample nucleic acid both using the agent for raising the melting temperature of a double-stranded nucleic acid; and a nucleic acid mismatch detection kit and a mutation detection kit including the agent for raising the melting temperature of a double-stranded nucleic acid and the method for detecting a nucleic acid mismatch or the method for detecting mutation.

### TECHNICAL FIELD

The present invention relates to an agent for raising a melting temperature (Tm) of a double-stranded nucleic acid, a method for detecting mismatching in a double-stranded nucleic acid or mutation with respect to a standard nucleic acid in a sample nucleic acid with the agent, and a kit for carrying out the method. More specifically, the present invention relates to an agent for raising a melting temperature (Tm) of a double-stranded nucleic acid, comprising an amphoteric copolymer having a specific structure, a method for detecting mismatching in a double-stranded nucleic acid or mutation with respect to a standard nucleic acid in a sample nucleic acid with the agent, and a kit for carrying out the method.

### BACKGROUND ART

The detection of mutations in a genomic nucleic acid has been widely performed to identify specific bacteria or viruses or to determine disease susceptibility, drug response in humans, and recently, it has been required to detect mutation at one base in a genomic nucleic acid accurately, rapidly, and at low cost.

Various methods for detecting mutation in a genomic nucleic acid have been developed, and for example, a method for detecting mismatching with respect to a standard nucleic acid in a sample nucleic acid has been proposed, wherein the method comprises allowing a double-stranded nucleic acid consisting of a standard nucleic acid and a complementary strand thereof to coexist with a sample nucleic acid, and measuring the speed or rate at which the complementary strand is replaced with the sample nucleic acid (Patent Literatures 1 to 3). In this method, a polymer obtained by graft-polymerizing a cationic polymer such as polylysine or polyarginine as the main chain, and dextran, polyethylene glycol, or the like as the side chain, is added to promote the rate at which a complementary strand is replaced with a sample nucleic acid, thereby enhancing the detection sensitivity It is thus stated that mismatching even at one-base level can be detected. However, this method requires fluorescence resonance energy transfer (FRET) for detection of the replacement of a complementary strand with a sample nucleic acid, and thus requires labeling the standard nucleic acid and the complementary strand with a fluorescent dye. If this is not necessary, it is more convenient.

As a method that can detect mutation in a genomic nucleic acid without a fluorescent dye-labeled nucleic acid, e.g. for FRET, there are methods of detecting mismatching in a double-stranded nucleic acid with a melting temperature (Tm).

The melting temperature (Tm) means a temperature at which 50% of a double-stranded nucleic acid denatures into single strands, and the melting temperature (Tm) is lower in a mismatch double strand having any un-complementary relation as compared with a full-match double strand having complementary relation. Accordingly, mutation in a sample nucleic acid with respect to a standard nucleic acid can be detected by utilizing this phenomenon, or a target sequence can be detected through a difference in nucleic acid amplification efficiency caused by this phenomenon. However, a change in a melting temperature (Tm) caused by mismatching at one base is considered about 1 to 3° C. Therefore, in order to use a melting temperature (Tm) to detect very few mutations in a genomic nucleic acid, it is necessary to improve the sensitivity.

In this regard, methods that analyze a melting curve to make slight mutation detectable have been developed (Patent Literatures 4 and 5). However, these methods require strict temperature control and thus require high-precision thermostatic devices. Therefore, a simpler method is desired for practical use.

Patent Literature 1 described above also describe that Tm values of a 20-mer double-stranded nucleic acid having mismatching at one base and a full-match 20-mer double-stranded nucleic acid were measured in the presence and absence of the above-mentioned cationic polymers, and shows that a Tm rise value due to the presence of each cationic polymer. Unfortunately, the Tm rise values of the mismatch and full-match double-stranded nucleic acids due to the presence of the cationic polymer are both about 15° C., and there is no significant difference between them.

### CITATION LIST

### Patent Literature

PATENT LITERATURE 1: WO 03/018841 A1
PATENT LITERATURE 2: JP-A-2001-78769
PATENT LITERATURE 3: JP-A-2008-278779
PATENT LITERATURE 4: JP-A-2005-58107
PATENT LITERATURE 5: JP-A-2003-528626
PATENT LITERATURE 6: WO 2016/167320 A1
PATENT LITERATURE 7: JP-B-6242336
PATENT LITERATURE 8: JP-B-5813263
PATENT LITERATURE 9: JP-B-4731324
PATENT LITERATURE 10: JP-B-4383178
PATENT LITERATURE 11: JP-B-5030998
PATENT LITERATURE 12: JP-B-4151751
PATENT LITERATURE 13: JP-A-2001-89496
PATENT LITERATURE 14: WO 2003/068795
PATENT LITERATURE 15: WO 2005/021570
PATENT LITERATURE 16: JP-B-3756313

### SUMMARY OF INVENTION

### Technical Problem

An object of the present invention is to provide a novel agent for raising a melting temperature (Tm) of a double-stranded nucleic acid, a method for detecting mismatching in a double-stranded nucleic acid or mutation in a sample nucleic acid with respect to a standard nucleic acid, and a kit for carrying out the method, which can address the problems in the prior art as described above.

### Solution to Problem

As a result of intensive studies, the present inventors have found that when an amphoteric copolymer comprising a specific cationic constituent unit (1) and a specific anionic constituent unit (2) (hereinafter, may also be abbreviated as “specific amphoteric copolymer”) is added to a nucleic acid solution, the presence of the specific amphoteric copolymer increases a difference between a Tm rise value of a full-match double-stranded nucleic acid having a complementary relation and a Tm rise value of a mismatch double-stranded nucleic acid having an un-complementary relation, which enables detection of mismatching even at one-base level, thus leading the completion of the present invention.

That is, the present invention provides the following agent for raising a melting temperature, method for detecting nucleic acid mismatching, method for detecting mutation in a sample nucleic acid with respect to a standard nucleic acid, kit for detecting nucleic acid mismatching, and kit for detecting mutation.

[1] An agent for raising a melting temperature of a double-stranded nucleic acid, comprising an amphoteric copolymer, wherein the amphoteric copolymer comprises:
at least one type of cationic constituent unit (1) selected from the group consisting of
a constituent unit (1-1) having a structure represented by general formula (I-a) or general formula (I-b), or a structure of an acid addition salt thereof:

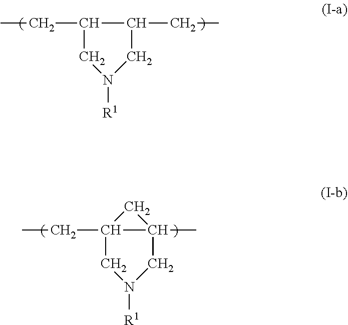

wherein R¹ represents a hydrogen atom, an alkyl group having 1 to 10 carbon atoms that may have a hydroxyl group, a cycloalkyl group having 5 to 10 carbon atoms, or an aralkyl group having 7 to 10 carbon atoms,
a constituent unit (1-2) having a structure represented by general formula (I-c) or general formula (I-d):

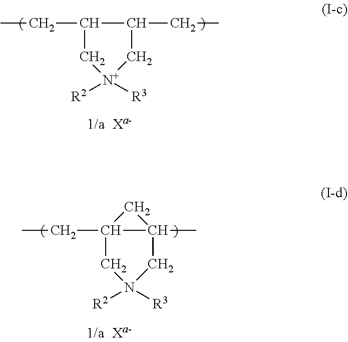

wherein R² and R³ each independently represent a hydrogen atom, an alkyl group having 1 to 10 carbon atoms that may have a hydroxyl group, a cycloalkyl group having 5 to 10 carbon atoms, or an aralkyl group having 7 to 10 carbon atoms, X^{a-} represents a counter ion, and a represents the valency number of the counter ion,
a constituent unit (1-3) having a structure represented by general formula (1-e), or a structure of an acid addition salt thereof:

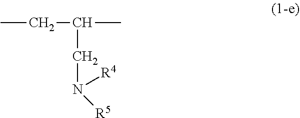

wherein R⁴ and R⁵ each independently represent a hydrogen atom, an alkyl group having 1 to 12 carbon atoms that may have a hydroxyl group, an aralkyl group having 7 to 10 carbon atoms, or a cycloalkyl group having 5 to 6 carbon atoms, and
a constituent unit (1-4) having a structure represented by general formula (1-f), or a structure of an acid addition salt thereof:

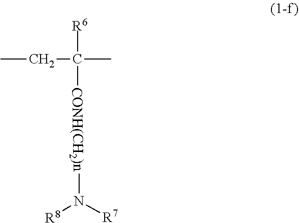

wherein R⁶ represents a hydrogen atom or a methyl group, R⁷ and R⁸ each independently represent an alkyl group having 1 to 4 carbon atoms, and n is an integer of 2 to 4; and
at least one type of anionic constituent unit (2) selected from the group consisting of
a constituent unit (2-1) having a structure represented by general formula (II-a):

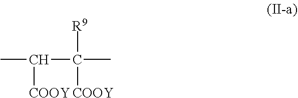

wherein R⁹ is a hydrogen or a methyl group, Y represents a hydrogen, Na, K, NH₄, ½Ca, ½Mg, ½Fe, ⅓Al, or ⅓Fe independently for each carboxy group to be bonded,
a constituent unit (2-2) having a structure represented by general formula (II-b):

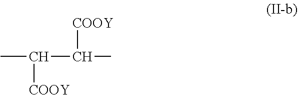

wherein Y represents a hydrogen, Na, K, NH₄, ½Ca, ½Mg, ½Fe, ⅓Al, or ⅓Fe independently for each carboxy group to be bonded,
a constituent unit (2-3) having a structure represented by general formula (II-c):

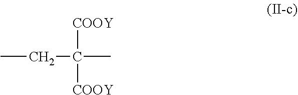

wherein Y represents a hydrogen, Na, K, NH₄, ½Ca, ½Mg, ½Fe, ⅓Al, or ⅓Fe independently for each carboxy group to be bonded, and
a constituent unit (2-4) having a structure represented by general formula (II-d)

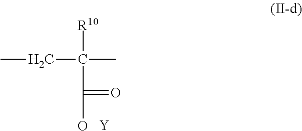

wherein R¹⁰ is a hydrogen or a methyl group, Y represents a hydrogen, Na, K, NH₄, ½Ca, ½Mg, ½Fe, ⅓Al, or ⅓Fe independently for each carboxy group to be bonded.

[2] The agent for raising a melting temperature of a double-stranded nucleic acid according to [1], wherein
the cationic constituent unit (1) is selected from the group consisting of the constituent unit (1-1) and the constituent unit (1-4), and
the anionic constituent unit (2) is selected from the group consisting of the constituent unit (2-1) and the constituent unit (2-4).

[3] The agent for raising a melting temperature of a double-stranded nucleic acid according to [1] or [2], wherein the amphoteric copolymer further comprises a nonionic constituent unit (3).

[4] A method for detecting nucleic acid mismatching between a sample nucleic acid and a probe nucleic acid, comprising the steps of:
(1) measuring melting temperatures of a double-stranded nucleic acid between the probe nucleic acid and a nucleic acid having a base sequence complementary to the probe nucleic acid with and without adding the agent for raising a melting temperature of a double-stranded nucleic acid according to any one of [1] to [3], and determining a rise value between the melting temperatures of the probe nucleic acid and the nucleic acid having a base sequence complementary to the probe nucleic acid due to the presence of the amphoteric copolymer (standard ΔTm);
(2) measuring melting temperatures of a double-stranded nucleic acid between the sample nucleic acid and the probe nucleic acid with and without adding the agent for raising a melting temperature according to any one of [1] to [3], and determining a rise value between the melting temperatures of the double-stranded nucleic acid of the sample nucleic acid and the probe nucleic acid due to the presence of the amphoteric copolymer (sample ΔTm); and
(3) determining the presence or absence of nucleic acid mismatch based on a value (ΔΔTm) obtained by subtracting the sample ΔTm from the standard ΔTm.

[5] The method for detecting nucleic acid mismatching according to [4], wherein it is determined that a nucleic acid mismatch is present when the ΔΔTm is 5.0° C. or more.

[6] A kit for the detection of nucleic acid mismatching between a sample nucleic acid and a probe nucleic acid, comprising:
the probe nucleic acid;
a nucleic acid having a base sequence complementary to the probe nucleic acid; and
the agent for raising a melting temperature according to any one of [1] to [3].

[7] A method for detecting mutation between a sample nucleic acid and a standard nucleic acid, comprising the steps of:
(1) measuring melting temperatures of a double-stranded nucleic acid between the standard nucleic acid and a nucleic acid having a base sequence complementary to the standard nucleic acid with and without adding the agent for raising a melting temperature according to any one of [1] to [3], and determining a rise value (standard ΔTm) between the melting temperatures of the double-stranded nucleic acid between the standard nucleic acid and the nucleic acid having the base sequence complementary to the standard nucleic acid due to the presence of the amphoteric copolymer;
(2) measuring melting temperatures of a double-stranded nucleic acid of the sample nucleic acid and the nucleic acid having a base sequence complementary to the standard nucleic acid with and without adding the agent for raising a melting temperature according to any one of [1] to [3], and determining a rise value (sample ΔTm) between the melting temperatures of the double-stranded nucleic acid between the sample nucleic acid and the nucleic acid having the base sequence complementary to the standard nucleic acid due to the presence of the amphoteric copolymer; and
(3) determining the presence or absence of mutation based on a value (ΔΔTm) obtained by subtracting the sample ΔTm from the standard ΔTm.

[8] The method for detecting mutation according to [7], wherein it is determined that mutation is present when the ΔΔTm is 5.0° C. or more.

[9] A kit for the detection of mutation between a sample nucleic acid and a standard nucleic acid, comprising:
the standard nucleic acid;
a nucleic acid having a base sequence complementary to the standard nucleic acid; and
the agent for raising a melting temperature according to any one of [1] to [3].

[10] Use of an amphoteric copolymer for use in producing an agent for raising a melting temperature of a double-stranded nucleic acid, wherein the amphoteric copolymer comprises:
at least one type of cationic constituent unit (1) selected from the group consisting of
a constituent unit (1-1) having a structure represented by general formula (I-a) or general formula (I-b), or a structure of an acid addition salt thereof:

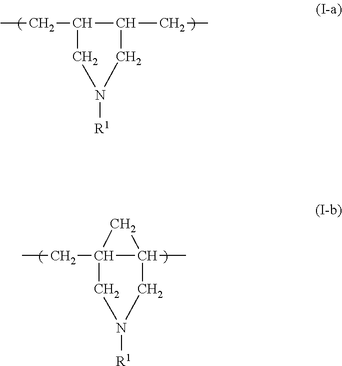

wherein R¹ represents a hydrogen atom, an alkyl group having 1 to 10 carbon atoms that may have a hydroxyl group, a cycloalkyl group having 5 to 10 carbon atoms, or an aralkyl group having 7 to 10 carbon atoms,
a constituent unit (1-2) having a structure represented by general formula (I-c) or general formula (I-d):

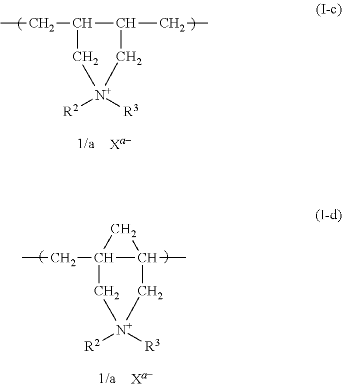

wherein R² and R³ each independently represent a hydrogen atom, an alkyl group having 1 to 10 carbon atoms that may have a hydroxyl group, a cycloalkyl group having 5 to 10 carbon atoms, or an aralkyl group having 7 to 10 carbon atoms, X^{a-} represents a counter ion, and a represents the valency number of the counter ion,
a constituent unit (1-3) having a structure represented by general formula (1-e) or a structure of an acid addition salt thereof:

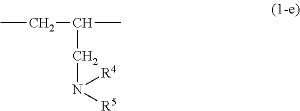

wherein R⁴ and R⁵ each independently represent a hydrogen atom, an alkyl group having 1 to 12 carbon atoms that may have a hydroxyl group, an aralkyl group having 7 to 10 carbon atoms, or a cycloalkyl group having 5 to 6 carbon atoms, and
a constituent unit (1-4) having a structure represented by general formula (1-f) or a structure of an acid addition salt thereof:

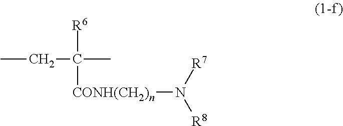

wherein R⁶ represents a hydrogen atom or a methyl group, R⁷ and R⁸ each independently represent an alkyl group having 1 to 4 carbon atoms, and n is an integer of 2 to 4; and
at least one type of anionic constituent unit (2) selected from the group consisting of
a constituent unit (2-1) having a structure represented by general formula (II-a):

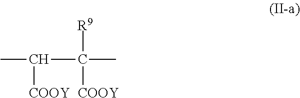

wherein R⁹ is a hydrogen atom or a methyl, Y represents a hydrogen, Na, K, NH₄, ½Ca, ½Mg, ½Fe, ⅓Al, or ⅓Fe independently for each carboxy group to be bonded,
a constituent unit (2-2) having a structure represented by general formula (II-b):

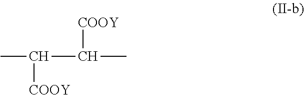

wherein Y represents a hydrogen, Na, K, NH₄, ½Ca, ½Mg, ½Fe, ⅓Al, or ⅓Fe independently for each carboxy group to be bonded,
a constituent unit (2-3) having a structure represented by general formula (II-c):

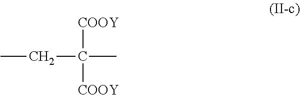

wherein Y represents a hydrogen, Na, K, NH₄, ½Ca, ½Mg, ½Fe, ⅓Al, or ⅓Fe independently for each carboxy group to be bonded, and
a constituent unit (2-4) having a structure represented by general formula (II-d):

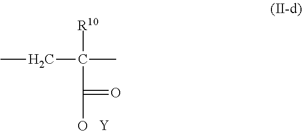

wherein R¹⁰ is a hydrogen or a methyl group, Y represents a hydrogen, Na, K, NH₄, ½Ca, ½Mg, ½Fe, ⅓Al, or ⅓Fe independently for each carboxy group to be bonded.

[11] The use according to [10], wherein
the cationic constituent unit (1) is selected from the group consisting of the constituent unit (1-1) and the constituent unit (1-4), and
the anionic constituent unit (2) is selected from the group consisting of the constituent unit (2-1) and the constituent unit (2-4).

[12] The use according to [10] or [11], wherein
the amphoteric copolymer further comprises a nonionic constituent unit (3).

[13] A method for raising a melting temperature of a double-stranded nucleic acid, comprising mixing an amphoteric copolymer with the double-stranded nucleic acid in a solution, wherein the amphoteric copolymer comprises:
at least one type of cationic constituent unit (1) selected from the group consisting of
a constituent unit (1-1) having a structure represented by general formula (I-a) or general formula (I-b) or a structure of an acid addition salt thereof:

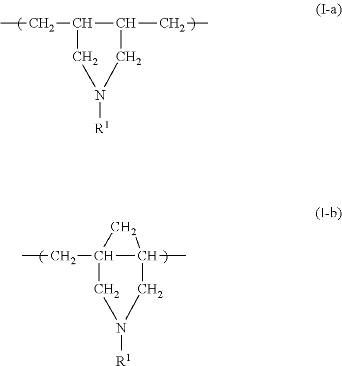

wherein R¹ represents a hydrogen atom, an alkyl group having 1 to 10 carbon atoms that may have a hydroxyl group, a cycloalkyl group having 5 to 10 carbon atoms, or an aralkyl group having 7 to 10 carbon atoms,
a constituent unit (1-2) having a structure represented by general formula (I-c) or general formula (I-d)

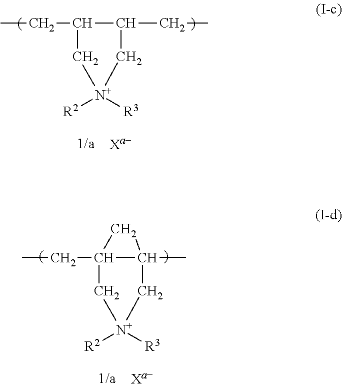

wherein R² and R³ each independently represent a hydrogen atom, an alkyl group having 1 to 10 carbon atoms that may have a hydroxyl group, a cycloalkyl group having 5 to 10 carbon atoms, or an aralkyl group having 7 to 10 carbon atoms, X^{a-} represents a counter ion, and a represents the valency number of the counter ion,
a constituent unit (1-3) having a structure represented by general formula (1-e) or a structure of an acid addition salt thereof:

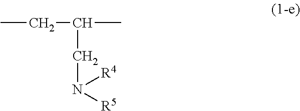

wherein R⁴ and R⁵ each independently represent a hydrogen atom, an alkyl group having 1 to 12 carbon atoms that may have a hydroxyl group, an aralkyl group having 7 to 10 carbon atoms, or a cycloalkyl group having 5 to 6 carbon atoms, and
a constituent unit (1-4) having a structure represented by general formula (1-f) or a structure of an acid addition salt thereof:

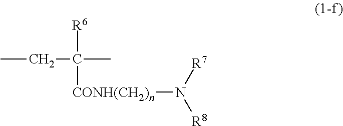

wherein R⁶ represents a hydrogen atom or a methyl group, R⁷ and R⁸ each independently represent an alkyl group having 1 to 4 carbon atoms, and n is an integer of 2 to 4; and
at least one type of anionic constituent unit (2) selected from the group consisting of
a constituent unit (2-1) having a structure represented by general formula (II-a):

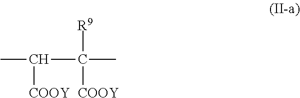

wherein R⁹ is a hydrogen or a methyl group, Y represents a hydrogen, Na, K, NH₄, ½Ca, ½Mg, ½Fe, ⅓Al, or ⅓Fe independently for each carboxy group to be bonded,
a constituent unit (2-2) having a structure represented by general formula (II-b):

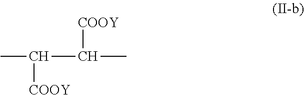

wherein Y represents a hydrogen, Na, K, NH₄, ½Ca, ½Mg, ½Fe, ⅓Al, or ⅓Fe independently for each carboxy group to be bonded,
a constituent unit (2-3) having a structure represented by general formula (II-c):

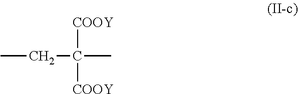

wherein Y represents a hydrogen, Na, K, NH₄, ½Ca, ½Mg, ½Fe, ⅓Al, or ⅓Fe independently for each carboxy group to be bonded, and
a constituent unit (2-4) having a structure represented by general formula (II-d):

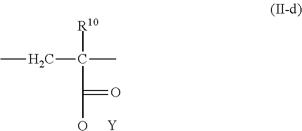

wherein R¹⁰ is a hydrogen or a methyl group, Y represents a hydrogen, Na, K, NH₄, ½Ca, ½Mg, ½Fe, ⅓Al, or ⅓Fe independently for each carboxy group to be bonded.

[14] The method according to [13], wherein
the cationic constituent unit (1) is selected from the group consisting of the constituent unit (1-1) and the constituent unit (1-4), and
the anionic constituent unit (2) is selected from the group consisting of the constituent unit (2-1) and the constituent unit (2-4).

[15] The method according to [13] or [14], wherein
the amphoteric copolymer further comprises a nonionic constituent unit (3).

According to the present invention, mismatching of a double-stranded nucleic acid can be detected simply and with high sensitivity by using a melting temperature (Tm) of the double-stranded nucleic acid. Therefore, the presence or absence of mutation in a sample nucleic acid with respect to a standard nucleic acid (for example, wild-type genomic nucleic acid) can be detected simply and with high sensitivity by using a melting temperature (Tm) of a double-stranded nucleic acid.

### BRIEF DESCRIPTION OF DRAWINGS

FIG. 1 shows melting curves of a double-stranded between a standard nucleic acid oligomer and a complementary strand oligomer in the presence and absence of a dimethylamino propyl methacrylamide hydrochloride/acrylamide/acrylic acid copolymer, and in the presence of MgCl₂ in place of the copolymer.

FIG. 2 shows melting curves of a double-stranded between a nucleic acid oligomer having mutation at one base with respect to a standard nucleic acid oligomer and an oligomer strand fully complementary to the standard nucleic acid oligomer in the presence and absence of a dimethylamino propyl methacrylamide hydrochloride/acrylamide/acrylic acid copolymer, and in the presence of MgCl₂ in place of the copolymer.

FIG. 3 shows melting curves of a double-stranded between a standard nucleic acid oligomer and a complementary strand oligomer in the presence and absence of a diallylamine hydrochloride/maleic acid copolymer, and in the presence of MgCl₂ in place of the copolymer.

FIG. 4 shows melting curves of a double-stranded between a nucleic acid oligomer having mutation at one base with respect to a standard nucleic acid oligomer and an oligomer strand fully complementary to the standard nucleic acid oligomer in the presence and absence of a diallylamine hydrochloride/maleic acid copolymer, and in the presence of MgCl₂ in place of the copolymer.

FIG. 5 shows melting curves of a double-stranded between a standard nucleic acid oligomer and a complementary strand oligomer in the presence and absence of a diallylmethylamine/maleic acid copolymer, and in the presence of MgCl₂ in place of the copolymer.

FIG. 6 shows melting curves of a double-stranded between a nucleic acid oligomer having mutation at one base with respect to a standard nucleic acid oligomer and an oligomer strand fully complementary to the standard nucleic acid oligomer in the presence and absence of a diallylmethylamine/maleic acid copolymer, and in the presence of MgCl₂ in place of the copolymer.

FIG. 7 shows melting curves of a double-stranded between a standard nucleic acid oligomer and a complementary strand oligomer in the presence or absence of a diallylamine hydrochloride/acrylamide/acrylic acid copolymer, and in the presence of MgCl₂ in place of the copolymer.

FIG. 8 shows melting curves of a double-stranded between a nucleic acid oligomer having mutation at one base with respect to a standard nucleic acid oligomer and an oligomer strand complementary to the standard nucleic acid oligomer in the presence and absence of a diallylamine hydrochloride/acrylamide/acrylic acid copolymer, and in the presence of MgCl₂ in place of the copolymer.

FIG. 9 shows melting curves of a double-stranded between a standard nucleic acid oligomer and a fully complementary strand oligomer in the presence and absence of an allylamine polymer (weight average molecular weight (Mw): 3000), and in the presence of MgCl₂ in place of the copolymer.

FIG. 10 shows melting curves of a double-stranded between a nucleic acid oligomer having mutation at one base with respect to a standard nucleic acid oligomer and an oligomer strand complementary to the standard nucleic acid oligomer in the presence and absence of an allylamine polymer (weight average molecular weight (Mw): 3000), and in the presence of MgCl₂ in place of the copolymer.

FIG. 11 shows melting curves of a double-stranded between a standard nucleic acid oligomer and a fully complementary strand oligomer in the presence and absence of an allylamine polymer (weight average molecular weight (Mw): 8000), and in the presence of MgCl₂ in place of the copolymer.

FIG. 12 shows melting curves of a double-stranded between a nucleic acid oligomer having a mutation at one base with respect to a standard nucleic acid oligomer and an oligomer strand complementary to the standard nucleic acid oligomer in the presence and absence of an allylamine polymer (weight average molecular weight (Mw): 8000), and in the presence of MgCl₂ in place of the copolymer.

FIG. 13 shows melting curves of a double-stranded between a standard nucleic acid oligomer and a complementary strand oligomer in the presence and absence of an allylamine polymer (weight average molecular weight (Mw): 15000), and in the presence of MgCl₂ in place of the copolymer.

FIG. 14 shows melting curves of a double-stranded between a nucleic acid oligomer having a mutation at one base with respect to a standard nucleic acid oligomer and an oligomer strand fully complementary to the standard nucleic acid oligomer in the presence and absence of an allylamine polymer (weight average molecular weight (Mw): 15000), and in the presence of MgCl₂ in place of the copolymer.

FIG. 15 shows melting curves of a double-stranded between a standard nucleic acid oligomer and a complementary strand oligomer in the presence and absence of an allylamine hydrochloride/diallylamine hydrochloride copolymer (weight average molecular weight (Mw): 100000), and in the presence of MgCl₂ in place of the copolymer.

FIG. 16 shows melting curves of a double-stranded between a nucleic acid oligomer having mutation at one base with respect to a standard nucleic acid oligomer and an oligomer strand fully complementary to a standard nucleic acid oligomer in the presence and absence of an allylamine hydrochloride/diallylamine hydrochloride copolymer (weight average molecular weight (Mw): 100000), and in the presence of MgCl₂ in place of the copolymer.

FIG. 17 shows melting curves of a double-stranded between a standard nucleic acid oligomer and a complementary strand oligomer in the presence and absence of an allylamine hydrochloride/diallylamine hydrochloride copolymer (weight average molecular weight (Mw): 20000), and in the presence of MgCl₂ in place of the copolymer.

FIG. 18 shows melting curves of a double-stranded between a nucleic acid oligomer having a mutation at one base with respect to a standard nucleic acid oligomer and an oligomer strand fully complementary to a standard nucleic acid oligomer in the presence and absence of an allylamine hydrochloride/diallylamine hydrochloride copolymer (weight average molecular weight (Mw): 20000), and in the presence of MgCl₂ in place of the copolymer.

FIG. 19 shows melting curves of a double-stranded between a standard nucleic acid oligomer and a complementary strand oligomer in the presence and absence of 50 mol % acetylated polyallylamine (weight average molecular weight (Mw):15000), and in the presence of MgCl₂ in place of the copolymer.

FIG. 20 shows melting curves of a double-stranded between a nucleic acid oligomer having a mutation at one base with respect to a standard nucleic acid oligomer and an oligomer strand fully complementary to the standard nucleic acid oligomer in the presence and absence of 50 mol % acetylated polyallylamine (weight average molecular weight (Mw): 15000), and in the presence of MgCl₂ in place of the copolymer.

FIG. 21 shows melting curves of a double-stranded between a standard nucleic acid oligomer and a complementary strand oligomer in the presence and absence of polyacrylamide, and in the presence of MgCl₂ in place of the copolymer.

FIG. 22 shows melting curves of a double-stranded between a nucleic acid oligomer having a mutation at one base with respect to a standard nucleic acid oligomer and an oligomer strand fully complementary to the standard nucleic acid oligomer in the presence and absence of polyacrylamide, and in the presence of MgCl₂ in place of the copolymer.

FIG. 23 shows melting curves of a double-stranded between a standard nucleic acid oligomer and a BNA (bridged nucleic acid) complementary strand oligomer in the presence and absence of a dimethylamino propyl methacrylamide hydrochloride/acrylamide/acrylic acid copolymer.

FIG. 24 shows melting curves of a double-stranded between a nucleic acid oligomer having a mutation at one base with respect to a standard nucleic acid oligomer and a BNA oligomer strand complementary to the standard nucleic acid oligomer in the presence and absence of a dimethylamino propyl methacrylamide hydrochloride/acrylamide/acrylic acid copolymer.

FIG. 25 shows melting curves of a double-stranded between a standard nucleic acid oligomer and a BNA complementary strand oligomer in the presence and absence of a diallylamine hydrochloride/maleic acid copolymer.

FIG. 26 shows melting curves of a double-stranded melting curve between a nucleic acid oligomer having a mutation at one base with respect to a standard nucleic acid oligomer and a BNA oligomer strand complementary to the standard nucleic acid oligomer in the presence and absence of a diallylamine hydrochloride/maleic acid copolymer.

### DESCRIPTION OF EMBODIMENTS

The embodiments of the present invention will be described in detail. However, the present invention should not be limited to the following embodiments.

The present invention relates to an agent for raising a melting temperature (Tm) of a double-stranded nucleic acid comprising an amphoteric copolymer having a specific constituent unit, a method for detecting mismatching in a double-stranded nucleic acid or mutation with respect to a standard nucleic acid in a sample nucleic acid with the agent, and a kit for carrying out the method. Hereinafter, the embodiments of the present invention will be described in detail.

### 1. Agent for Raising Melting Temperature (Tm) of Double-Stranded Nucleic Acid

One embodiment of the present invention relates to an agent for raising a Tm of a double-stranded nucleic acid comprising a specific amphoteric copolymer. The agent for raising a Tm of a double-stranded nucleic acid may consist of a specific amphoteric copolymer, or may comprise a component other than the specific amphoteric copolymer, as in the case of an aqueous solution obtained by dissolving the specific amphoteric copolymer in a nuclease-free medium (for example, water).

The agent for raising a Tm of a double-stranded nucleic acid raises a melting temperature of a double-stranded nucleic acid by 16.0° C. or more, and preferably by 20.0° C. or more. In particular, in a case where the double-stranded nucleic acid is a double-stranded nucleic acid containing no mismatching (full-match double-stranded nucleic acid), the agent for raising a Tm of a double-stranded nucleic acid raises a melting temperature of the full-match double-stranded nucleic acid by 20.0° C. or more, preferably 25.0° C. or more, and particularly preferably 30.0° C. or more.

### Specific Amphoteric Copolymer

A specific amphoteric copolymer comprises a cationic constituent unit (1) having a specific structure and an anionic constituent unit (2) having a specific structure, and the specific amphoteric copolymer may consist of the cationic constituent unit (1) and the anionic constituent unit (2), or may comprise other constituent units in addition to the cationic constituent unit (1) and the anionic constituent unit (2). Examples of the other constituent units include a nonionic constituent unit (3) described later, and a cationic or anionic constituent unit having a structure, which does not correspond to any of the cationic constituent unit (1) having a specific structure and the anionic constituent unit (2) having a specific structure.

### Cationic Constituent Unit (1)

A cationic constituent unit (1) constituting a specific amphoteric copolymer is at least one type of cationic constituent unit selected from the group consisting of the following constituent units (1-1), (1-2), (1-3), and (1-4).

As shown below using general formulas or the like, the cationic constituent unit (1) has an amino group as the cationic group in the structure, and from the viewpoint of realizing a favorable Tm-value rising effect, and the like, the amino group is preferably a secondary or tertiary amino group.

The specific amphoteric copolymer may comprise only one type of cationic constituent unit (1), or may comprise two or more types of cationic constituent units (1). The two or more types of cationic constituent units (1) when the two or more types of cationic constituent units (1) are to be contained may be a combination of constituent units that are all classified as constituent units (1-1), a combination of constituent units that are all classified as constituent units (1-2), a combination of constituent units that are all classified as constituent units (1-3), or a combination of constituent units that are all classified as constituent units (1-4), or may be a combination of constituent units that are classified as different from each other among the constituent units (1-1) to (1-4).

In the specific amphoteric copolymer, since mismatching in a double-stranded nucleic acid or mutation with respect to a standard nucleic acid in a sample nucleic acid is more reliably detectable, the cationic constituent unit (1) is particularly preferably selected from the group consisting of constituent units (1-1) and (1-4), and the cationic constituent unit (1) is most preferably the constituent unit (1-1).

### Constituent Unit (1-1)

A constituent unit (1-1) is a constituent unit having a structure represented by the following general formula (I-a) or (I-b), or a structure of an acid addition salt thereof:

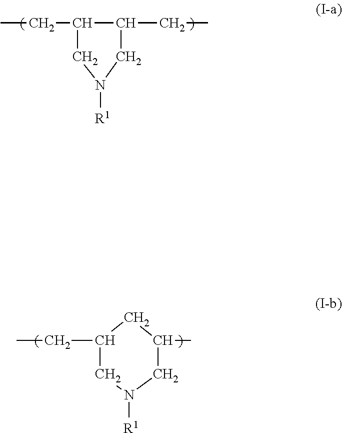

In the formula, R¹ represents a hydrogen atom, an alkyl group having 1 to 10 carbon atoms that may have a hydroxyl group, a cycloalkyl group having 5 to 10 carbon atoms, or an aralkyl group having 7 to 10 carbon atoms. R¹ represents preferably a hydrogen atom, a methyl group, an ethyl group, or a benzyl group, and particularly preferably a hydrogen atom, or a methyl group.

The constituent unit (1-1) may have a structure that is an inorganic acid salt, an organic acid salt, or the like of the structure represented by the above structural formula (1-a) or (1-b), that is, a structure of an acid addition salt thereof.

In a case where the specific amphoteric copolymer has a constituent unit (1-1), from the viewpoint of production costs, and the like, a diallylamine monomer having an addition salt is preferably used in producing the specific amphoteric copolymer. Since a process of removing the addition salt such as HCl from the polymer is complicated and causes an increase in cost, it is a preferable embodiment also from the viewpoint of costs and the like to use an addition salt-type constituent unit (1-1) with which the specific amphoteric copolymer can be produced without such a process.

From the viewpoint of the ease of availability, the controllability of reaction, and the like, the inorganic acid salt or organic acid salt in the constituent unit (1-1) of this embodiment is preferably a hydrochloride, a carboxylate, a sulfonate, or an alkyl sulfate, and particularly preferably a hydrochloride.

### Constituent Unit (1-2)

A constituent unit (1-2) is a constituent unit having a structure represented by the following general formula (I-c) or (I-d):

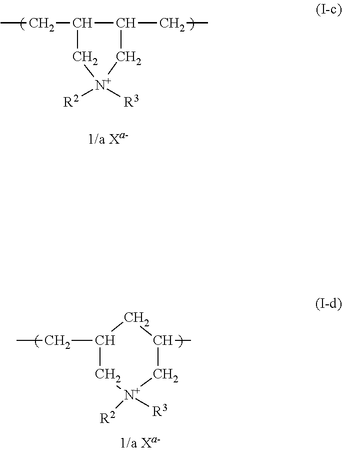

In the formula, R² and R³ each independently represent a hydrogen atom, an alkyl group having 1 to 10 carbon atoms that may have a hydroxyl group, a cycloalkyl group having 5 to 10 carbon atoms, or an aralkyl group having 7 to 10 carbon atoms, X^{a-} represents a counter ion, and a represents the valency number of the counter ion.

R² and R³ each independently represent preferably a hydrogen atom, a methyl group, an ethyl group, or a benzyl group, and particularly preferably a methyl group, or an ethyl group.

From the viewpoint of realizing a favorable Tm-value rising effect, and the like, at least one of R² and R³ is preferably a hydrogen atom.

There is no particular limitation on the counter ion X^{a-}, but from the viewpoint of the ease of availability, the controllability of reaction, and the like, the counter ion X^{a-} is preferably a chloride ion, a carboxylic acid ion, a sulfonic acid ion, or an alkyl sulfate ion, and particularly preferably a chloride ion, or an ethyl sulfate ion.

From the viewpoint of production costs, and the like, a diallylamine monomer having a counter ion is preferably used in producing the specific amphoteric copolymer. Since a process of removing a counter ion from the polymer is complicated and causes an increase in cost, it is a preferable embodiment also from the viewpoint of costs and the like to use a specific amphoteric copolymer having a counter ion-type constituent unit (1-2) with which the specific amphoteric copolymer can be produced without such a process.

### Constituent Unit (1-3)

A constituent unit (1-3) is a constituent unit having a structure represented by the following general formula (I-e), or a structure of an acid addition salt thereof:

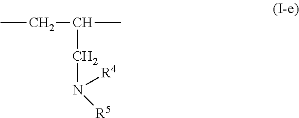

In the formula, R⁴ and R⁵ independently represent a hydrogen atom, an alkyl group having 1 to 12 carbon atoms that may have a hydroxyl group, an aralkyl group having 7 to 10 carbon atoms, or a cycloalkyl group having 5 to 6 carbon atoms.

The alkyl group having 1 to 12 carbon atoms or aralkyl group having 7 to 10 carbon atoms, which is preferable as R⁴ and R⁵, may be linear or branched. Examples of the group include a methyl group, an ethyl group, a n-propyl group, an isopropyl group, a n-butyl group, an isobutyl group, a sec-butyl group, a tert-butyl group, a pentyl group, a hexyl group, an octyl group, a decyl group, a dodecyl group, and a benzyl group. Further, examples of the cycloalkyl group having 5 to 6 carbon atoms, which is preferable as R⁴ and R⁵, include, but are not limited to, a cyclopentyl group, and a cyclohexyl group.

R⁴ and R⁵ each independently represent preferably a hydrogen atom, a methyl group, an ethyl group, or a benzyl group, and particularly preferably a hydrogen atom, or a methyl group.

From the viewpoint of realizing a favorable Tm value rising effect, and the like, both of R⁴ and R⁵ are not preferably hydrogen atoms at the same time.

There is no particular limitation on the type of an addition salt in a case where the constituent unit (1-3) is an acid addition salt having a structure represented by general formula (I-e), but from the viewpoint of the ease of availability, controllability of reaction, and the like, for example, a hydrochloride, a sulfate, a phosphate, a nitrate, a sulfite, a phosphite, a nitrite, a hydrobromide, an acetate, an amide sulfate, a methanesulfonate, a trifluoroacetate, a p-toluenesulfonate, or the like can be used.

Among them, a hydrochloride, a sulfate, a phosphate, and an amide sulfate are preferable, and a hydrochloride, a sulfate, a phosphate and an amide sulfate which have a structure derived from monoallylamine, are particularly preferable.

### Constituent Unit (1-4)

A constituent unit (1-4) is a constituent unit having a structure represented by the following general formula (I-f), or a structure of an acid addition salt thereof:

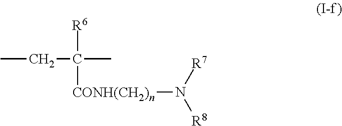

In the formula, R⁶ represents a hydrogen atom or a methyl group, R⁷ and R⁸ each independently represent an alkyl group having 1 to 4 carbon atoms, and n is an integer of 2 to 4.

R⁶ represents preferably a methyl group, n represents preferably 2 to 3, and R⁷ and R⁸ are each preferably a methyl group.

There is no particular limitation on the type of an addition salt in a case where the constituent unit (1-4) is an acid addition salt having a structure represented by general formula (I-f), but from the viewpoint of the ease of availability, controllability of reaction, and the like, for example, a hydrochloride, a sulfate, a phosphate, a nitrate, a sulfite, a phosphite, a nitrite, a hydrobromide, an acetate, an amide sulfate, a methanesulfonate, a trifluoroacetate, a p-toluenesulfonate, or the like can be used.

Among them, a hydrochloride, a sulfate, a phosphate, and an amide sulfate are preferable, and a hydrochloride, a sulfate, a phosphate and an amide sulfate which have a structure derived from monoallylamine, are particularly preferable.

The proportion of a cationic constituent unit (1) to the whole constituent units of the specific amphoteric copolymer is usually 10 to 70 mol %, preferably 15 to 60 mol %, and particularly preferably 20 to 55 mol %. In a case where two or more types of cationic constituent units (1) are contained, the proportion of the unit is defined based on the total amount of the two or more types of cationic constituent units (1).

### Anionic Constituent Unit (2)

An anionic constituent unit (2) constituting the specific amphoteric copolymer is at least one type of anionic constituent unit selected from the group consisting of the following constituent units (2-1), (2-2), (2-3), and (2-4).

The specific amphoteric copolymer may comprise only one type of anionic constituent unit (2), or may comprise two or more types of anionic constituent units (2). The two or more types of anionic constituent unit (2) when the two or more types of anionic constituent units (2) are to be contained may be a combination of constituent units that are all classified as constituent units (2-1), a combination of constituent units that are all classified as constituent units (2-2), a combination of constituent units that are all classified as constituent units (2-3), or a combination of constituent units that are all classified as constituent units (2-4), or may be a combination of constituent units that are classified as different from each other among the constituent units (2-1) to (2-4).

In the specific amphoteric copolymer, since mismatching in a double-stranded nucleic acid or mutation with respect to a standard nucleic acid in a sample nucleic acid is more reliably detectable, the anionic constituent unit (2) is particularly preferably selected from the group consisting of constituent units (2-1) and (2-4).

### Constituent Unit (2-1)

A constituent unit (2-1) is a constituent unit having a structure represented by the following general formula (II-a):

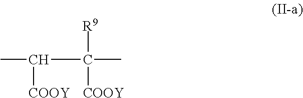

In the formula, R⁹ represents a hydrogen or a methyl group, Y represents a hydrogen, Na, K, NH₄, ½Ca, ½Mg, ½Fe, ⅓Al, or ⅓Fe independently for each carboxy group to be bonded.

R⁹ represents preferably a hydrogen, and Y represents preferably a hydrogen or Na. The constituent unit (2-1) is particularly preferably derived from maleic acid.

### Constituent Unit (2-2)

A constituent unit (2-2) is a constituent unit having a structure represented by the following general formula (II-b):

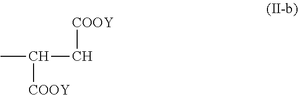

In the formula, Y represents a hydrogen, Na, K, NH₄, ½Ca, ½Mg, ½Fe, ⅓Al, or ⅓Fe independently for each carboxy group to be bonded.

Y represents preferably a hydrogen or Na.

### Constituent Unit (2-3)

A constituent unit (2-3) is a constituent unit having a structure represented by the following general formula (II-c), or a structure of an acid addition salt thereof.

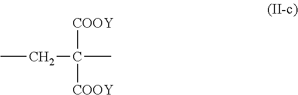

In the formula, Y represents a hydrogen, Na, K, NH₄, ½Ca, ½Mg, ½Fe, ⅓Al, or ⅓Fe independently for each carboxy group to be bonded.

Y represents preferably a hydrogen or Na.

### Constituent Unit (2-4)

A constituent unit (2-4) is a constituent unit having a structure represented by the following general formula (II-d):

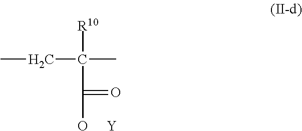

In the formula, R¹⁰ is a hydrogen or a methyl group, and Y represents a hydrogen, Na, K, NH₄, ½Ca, ½Mg, ½Fe, ⅓Al, or ⅓Fe independently for each carboxy group to be bonded.

R¹⁰ represents preferably a hydrogen, and Y represents preferably a hydrogen or Na.

The constituent unit (2-4) is preferably derived from (meth)acrylic acid, and particularly preferably derived from acrylic acid.

As the anionic constituent unit (2) in the specific amphoteric copolymer, only one type of anionic constituent unit (2) may be used alone, or multiple types of anionic constituent units (2) having structures different from each other may be used in combination.

In a case where multiple types of anionic constituent units that are different from each other are used, respective anionic constituent units may have structures different from each other within the range represented by the same general structural formula (II-a), (II-b), (II-c) or (II-d), or may have structures different from each other represented by different general structural formulas. In the former case, for example, multiple types of anionic constituent units (2), which are each represented by structural formula (II-a) but have structures different from each other due to the Y elements being different from each other, may be used. In the latter case, for example, one anionic constituent unit (2-1) having a structure represented by structural formula (II-a), and one anionic constituent unit (2-4) having a structure represented by structural formula (II-d) may be used.

The specific amphoteric copolymer comprises a cationic constituent unit (1) and an anionic constituent unit (2), and thus possesses cationicity and anionicity.

The specific amphoteric copolymer, which comprises a cationic constituent unit (1) having a specific structure and an anionic constituent unit (2) having a specific structure, significantly increases a difference in the melting temperature of a double-stranded nucleic acid between the presence and absence of double-stranded nucleic acid mismatch. Although not adhering to a theory, the mechanism for bringing about the remarkable effect may be due to as follows: If the cationic density of a cationic constituent unit(s) is too high, the bond between nucleic acids becomes too strong, which makes it difficult to measure a double-stranded nucleic acid melting temperature. On the other hand, if the cationic density of a cationic constituent unit(s) is too low, the rise in the melting temperature of a double-stranded nucleic acid becomes small and the detection sensitivity of mismatch decreases. It is thus inferred that the positive charge of a cationic constituent unit (1) having a specific structure and the negative charge of an anionic constituent unit (2) having a specific structure moderately interfere with each other, and the bond between nucleic acids is moderately strengthened.

As described above, since the mismatching in a double-stranded nucleic acid or the mutation with respect to a standard nucleic acid in a sample nucleic acid is more reliably detectable, in the specific amphoteric copolymer, the cationic constituent unit (1) is preferably selected from the group consisting of constituent units (1-1) and (1-4), and the anionic constituent unit (2) is preferably selected from the group consisting of constituent units (2-1) and (2-4), and thus, a specific amphoteric copolymer comprising a constituent unit selected from the group consisting of constituent units (1-1) and (1-4) as the cationic constituent unit (1) and a constituent unit selected from the group consisting of constituent units (2-1) and (2-4) as the anionic constituent unit (2) can be particularly preferably used.

The proportion of the total of the cationic constituent unit (1) and the anionic constituent unit (2) to the whole constituent units of a specific amphoteric copolymer is not particularly limited, but is usually 25 mol % or more, preferably 30 to 90 mol %, more preferably 35 to 75 mol %, and particularly preferably 40 to 60 mol %.

The proportion of the cationic constituent unit (1) and the anionic constituent unit (2) is also not particularly limited, but is usually 0.1:1 to 2:1, preferably 0.3:1 to 0.8:1, more preferably 0.4:1 to 1.2:1, and particularly preferably 0.5:1 to 1:1 in terms of molar ratio (cationic constituent unit (1):anionic constituent unit (2)).

In a case where the structures of the cationic constituent unit (1) and the anionic constituent unit (2) are known, the molar ratio of the cationic constituent unit (1) and anionic constituent unit (2) in the specific amphoteric copolymer can be specified by reprecipitating the specific amphoteric copolymer with an organic solvent such as isopropyl alcohol, or acetone, and by analyzing the reprecipitation in an appropriate mode according to the structure of the constituent unit, using Perkin Elmer 240011 CHNS/O fully automatic elemental analyzer or a device with a performance equivalent thereto. In addition, the measurement can be performed by using helium gas as a carrier gas, weighing a solid sample into a tin capsule, dropping the sample into a combustion tube to burn the sample at a combustion temperature of 1800° C. or more in pure oxygen gas, detecting each component to be measured by a frontal chromatography system with a separation column and a thermal conductivity detector, and quantifying the content of each element using a calibration factor. In this regard, in a case where the structures of the cationic constituent unit (1) and anionic constituent unit (2) in the specific amphoteric copolymer are unknown, the structures of the respective constituent units are specified by a known method using 1H-NMR or 13C-NMR before the measurement by the elemental analyzer. Further, the calculation can be performed with the amount of each monomer supplied in the production (copolymerization) of the specific amphoteric copolymer, and the amount of each monomer remaining without being incorporated into the specific amphoteric copolymer. In this regard, since the proportion (molar ratio) of the constituent unit derived from each monomer in the specific amphoteric copolymer is almost the same as the charge composition (molar ratio) of each constituent unit, the mixing ratio of a monomer may be treated as the proportion (molar ratio) of a constituent unit for convenience in the present specification.

### Other Constituent Units

The specific amphoteric copolymer may comprise other constituent units in addition to a cationic constituent unit (1) and an anionic constituent unit (2).

Examples of the other constituent units include a nonionic constituent unit (3), and a cationic or anionic constituent unit that does not correspond to any of the cationic constituent units (1) and the anionic constituent unit (2).

The specific amphoteric copolymer preferably comprises a nonionic constituent unit (3). A nonionic constituent unit (3) can be positioned between a cationic constituent unit (1) and an anionic constituent unit (2) to function as a spacer between the cationic constituent unit (1) and the anionic constituent unit (2) and control the interference between the positive and negative charges of each constituent unit, which allows a mismatch in a double-stranded nucleic acid or a mutation in a sample nucleic acid with respect to a standard nucleic acid to be more reliably detectable. Further, a nonionic constituent unit is preferably positioned between a cationic constituent unit (1) and an anionic constituent unit (2). In particular, the specific amphoteric copolymer particularly preferably comprises a nonionic constituent unit (3) in a case where a cationic constituent unit has a structure represented by general formula (I-f) or a structure of an acid addition salt thereof. In that case, a nonionic constituent unit particularly effectively functions as a spacer.

### Nonionic Constituent Unit (3)

A nonionic constituent unit (3) in the present embodiment may be a constituent unit derived from a nonionic monomer that can be copolymerized with a cationic constituent unit (1) and an anionic constituent unit (2), and is not otherwise particularly limited, but as the nonionic constituent unit (3), a constituent unit derived from a methacrylic acid ester-based monomer, an acrylic ester-based monomer, a methacrylamide-based monomer, an acrylamide-based monomer, sulfur dioxide, or the like can be preferably used. More specific examples of the nonionic constituent unit (3) include constituent units derived from methyl methacrylate, ethyl methacrylate, butyl methacrylate, methyl acrylate, ethyl acrylate, butyl acrylate, methacrylamide, N-methyl methacrylamide, dimethylmethacrylamide, N-(3-dimethylaminopropyl)methacrylamide, acrylamide, dimethylacrylamide, hydroxyethyl acrylamide, dimethylaminopropyl acrylamide, a dimethylaminopropyl acrylamide methyl chloride quaternary salt, acryloylmorpholine, isopropyl acrylamide, 4-t-butylcyclohexyl acrylate, and sulfur dioxide. It is particularly preferable to use a constituent unit derived from acrylamide.

The nonionic constituent unit (3) can usually be introduced into a polymer by using a nonionic monomer as the monomer.

There is no particular limitation on the content of the nonionic constituent unit (3) in a case where the specific amphoteric copolymer has the nonionic constituent unit (3), and the suitable amount varies depending on the type of the nonionic constituent unit (3), but the molar ratio with the anionic constituent unit (2), that is, nonionic constituent unit (3)/anionic constituent unit (2)=preferably 0.1/1 to 1/1, more preferably 0.2/1 to 0.8/1, furthermore preferably, 0.3/1 to 0.7/1, and particularly preferably 0.4/1 to 0.6/1.

In a case where the nonionic constituent unit (3) is derived from a methacrylic acid ester-based monomer, an acrylic ester-based monomer, a methacrylamide-based monomer, or an acrylamide-based monomer, the above molar ratio is preferably 0.1/1 to 1/1, more preferably 0.2/1 to 0.8/1, furthermore preferably, 0.3/1 to 0.7/1, and particularly preferably 0.4/1 to 0.6/1.

In a case where the nonionic constituent unit (3) is derived from sulfur dioxide, the above molar ratio is preferably 0.1/1 to 1/1, and particularly preferably 0.2/1 to 1/1.

### Production Method of Specific Amphoteric Copolymer

There is no particular limitation on the production method of a specific amphoteric copolymer, and the specific amphoteric copolymer can be produced by a method conventionally known in the technical field to which the invention belongs, and for example, the specific amphoteric copolymer can be produced by copolymerizing a cationic monomer having a structure corresponding to the cationic constituent unit (1), an anionic monomer having a structure corresponding to the anionic constituent unit (2), and if desired, a monomer such as a nonionic monomer having a structure corresponding to the nonionic constituent unit (3).

A solvent in a case of copolymerizing a cationic monomer having a structure corresponding to the cationic constituent unit (1), an anionic monomer having a structure corresponding to the anionic constituent unit (2), and the like is not particularly limited, and may be a water-based solvent, or an organic solvent such as alcohol, ether, sulfoxide, or amide, but is preferably a water-based solvent.

A concentration of the monomer in the case of copolymerizing a cationic monomer having a structure corresponding to the cationic constituent unit (1), an anionic monomer having a structure corresponding to the anionic constituent unit (2), and the like varies depending on the type of the monomer and the type of the solvent used for the copolymerization, but is usually 10 to 75% by mass in a case of a water-based solvent. This copolymerization reaction is usually a radical polymerization reaction, and is performed in the presence of a radical polymerization catalyst. The type of the radical polymerization catalyst is not particularly limited, and preferable examples of the radical polymerization catalyst include a peroxide such as t-butyl hydroperoxide, a persulfate such as ammonium persulfate, sodium persulfate, or potassium persulfate, and a water-soluble azo compound such as an azobis-based compound, or a diazo-based compound.

The amount of the radical polymerization catalyst to be added is commonly 0.1 to 20 mol %, and preferably 1.0 to 10 mol % based on the whole monomers. The polymerization temperature is commonly 0 to 100° C. and preferably 5 to 80° C., and the polymerization time is commonly 1 to 150 hours and preferably 5 to 100 hours. As the polymerization atmosphere, even in the air, there is no major problem in polymerization, and the polymerization can be performed in an atmosphere of an inert gas such as nitrogen.

### 2. Method for Detecting Nucleic Acid Mismatching Between Sample Nucleic Acid and Probe Nucleic Acid

Another embodiment of the present invention relates to a method for detecting nucleic acid mismatching between a sample nucleic acid and a probe nucleic acid, and the method comprises the steps of:
(1) determining a rise value between the melting temperatures of a double-stranded nucleic acid of the probe nucleic acid and a nucleic acid having a base sequence complementary to the probe nucleic acid due to the presence of the specific amphoteric copolymer (standard ΔTm);
(2) determining a rise value between the melting temperatures of a double-stranded nucleic acid of the sample nucleic acid and the probe nucleic acid due to the presence of the specific amphoteric copolymer (sample ΔTm); and
(3) determining the presence or absence of nucleic acid mismatching based on a value (ΔΔTm) obtained by subtracting the sample ΔTm from the standard ΔTm.

In step (1), the rise value (standard ΔTm) between the melting temperatures is determined by measuring a melting temperature (Tm1) of a double-stranded nucleic acid of the probe nucleic acid and a nucleic acid having a base sequence complementary to the probe nucleic acid in the presence of the specific amphoteric copolymer, and a melting temperature (Tm2) of a double-stranded nucleic acid of the probe nucleic acid and a nucleic acid having a base sequence complementary to the probe nucleic acid in the absence of the specific amphoteric copolymer, respectively, and by calculating Tm1−Tm2.

Similarly, in step (2), the rise value (sample ΔTm) between the melting temperatures is determined by measuring a melting temperature (Tm1) of a double-stranded nucleic acid of the sample nucleic acid and the probe nucleic acid in the presence of the specific amphoteric copolymer, and a melting temperature (Tm2) of a double-stranded nucleic acid of the sample nucleic acid and the probe nucleic acid in the absence of the specific amphoteric copolymer, and by calculating Tm1−Tm2.

In the specification of the present application, the term “nucleic acid” includes both DNA and RNA. The “nucleic acid” also includes an artificial nucleic acid. The sample nucleic acid and the nucleic acid having a sequence complementary to the probe nucleic acid may include any nucleic acid such as genomic DNA, cDNA, genomic RNA, mRNA, or rRNA. Further, the probe nucleic acid and the nucleic acid having a sequence complementary to the probe nucleic acid may be a natural nucleic acid or an artificial nucleic acid. The artificial nucleic acid is DNA or RNA in part of which an artificial nucleotide is contained, and examples of the artificial nucleotide include BNA (bridged nucleic acid), LNA (locked nucleic acid), PNA (peptide nucleic acid), a peptide nucleic acid (PHONA) having a phosphate group, and a nucleic acid in at least part of which a morpholino nucleic acid is contained (the details of these artificial nucleic acids are disclosed in, for example, Patent Literatures 6 to 16 and the like, and the contents are incorporated in the specification of the present application by standard).

The polymerization degree of the sample nucleic acid and the probe nucleic acid is not particularly limited, and is, from the point of detection sensitivity, usually 4 to 100 mers, preferably 5 to 50 mers, more preferably 6 to 30 mers, furthermore preferably 7 to 20 mers, and particularly preferably 8 to 15 mers.

The GC content of the sample nucleic acid and the probe nucleic acid is not particularly limited, and is, from the point of detection sensitivity, usually 30 to 80%, and preferably 50 to 75%.

The melting temperature of a double-stranded nucleic acid is usually measured by using a nucleic acid aqueous solution, and the solvent to be used may be a nuclease-free aqueous medium and is typically nuclease-free water. Further, the nucleic acid may be dissolved in a buffer solution, and as the buffer solution, for example, a buffer solution for nucleic acids used in a nucleic acid analysis reagent can be used. Further, the nucleic acid aqueous solution may contain a surfactant within the range of not affecting the rise in melting temperature (Tm) due to the specific amphoteric copolymer, and as the surfactant, a surfactant used in a nucleic acid analysis reagent can be mentioned.

In the measurement of the melting temperature (Tm) of a double-stranded nucleic acid, the specific amphoteric copolymer can be contained in a solution usually at a concentration of 0.03 to 3.0% by mass, and preferably at a concentration of 0.2 to 2.0% by mass.

In one embodiment of the present invention, in addition to the specific amphoteric copolymer, a known agent for raising a melting temperature (Tm) such as MgCl₂ may be contained in the reaction mixture. However, in a case of containing MgCl₂, the concentration is preferably 5 mM or less. Further, a salt such as NaCl may be contained within the range of not affecting the rise in melting temperature (Tm). In this case, the salt is preferably 300 mM or less.

In the measurement of the melting temperature (Tm) of a double-stranded nucleic acid, the sample nucleic acid and the probe nucleic acid each can be measured usually at a concentration of 10 to 1000 μM, and preferably at a concentration of 30 to 300 μM.

The solution pH can be set to 3.0 to 11.0, and preferably 5.0 to 9.0.

The temperature schedule in the measurement of a melting temperature (Tm) of a double-stranded nucleic acid varies depending on the method for detecting denaturation of the double-stranded nucleic acid, but basically, the temperature is allowed to gradually increase from the temperature at which the double strand is completely formed so that the denaturation of the double-stranded nucleic acid is caused.

Examples of the method for detecting the denaturation of a double-stranded nucleic acid include: a method of utilizing the fact that the absorbance of a nucleic acid in a UV range (for example, 260 nm) changes with the denaturation of the double-stranded nucleic acid; a method for detecting the degree of formation of the double-stranded nucleic acid by using a probe nucleic acid labeled with a fluorescent dye, enzyme, a light emitting dye, or the like, or a probe nucleic acid and a nucleic acid having a sequence complementary to the probe nucleic acid (for example, FRET method); and a method of utilizing the decrease in fluorescence intensity with the denaturation of the double-stranded nucleic acid by incorporating a fluorescent dye or the like between nucleic acids at the time of formation of the double-stranded nucleic acid (for example, intercalator method).

From the point that there is no need to prepare a probe nucleic acid labeled with a fluorescent dye or the like and real-time analysis is possible with a simple device, a method of utilizing the decrease in fluorescence intensity with the denaturation of the double-stranded nucleic acid by incorporating a fluorescent dye or the like between nucleic acids at the time of formation of the double-stranded nucleic acid (for example, intercalator method) is preferable. In addition, when referring to a specific numerical value of Tm in the specification of the present application, unless otherwise particularly specified by another method, the specific numerical value means a numerical value measured by the method described in Example 1, and specific numerical values of standard ΔTm, sample ΔTm, and ΔΔTm mean the values determined by calculation from the Tm thus obtained.

The detection sensitivity of nucleic acid mismatching between a sample nucleic acid and a probe nucleic acid may vary depending on conditions such as the length of the nucleic acid, the GC content of the nucleic acid, and the salt concentration, but in the detection method of the present invention, if the polymerization degree of the nucleic acid is 20 or less, even if there is mismatching of one base, the standard ΔTm is usually higher than the sample ΔTm by 5.0° C. or more. Therefore, in a case where the standard ΔTm is higher than the sample ΔTm by 5.0° C. or more, it is preferable to determine that double-stranded nucleic acid mismatching is present, and from the viewpoint of reducing false positives, preferably, in a case where the standard ΔTm is higher than the sample ΔTm by 7.5° C. or more, it is more preferable to determine that nucleic acid mismatching is present, and in a case where the standard ΔTm is higher than the sample ΔTm by 10.0° C. or more, it is particularly preferable to determine that nucleic acid mismatching is present.

3. Method for Detecting Mutation with Respect to Standard Nucleic Acid in Sample Nucleic Acid

As one embodiment of the method for detecting nucleic acid mismatching, the presence or absence of mutation in a sample nucleic acid with respect to a standard nucleic acid (for example, wild-type genomic nucleic acid) can be detected. Specifically, by using an antisense strand of a wild-type genomic nucleic acid as the probe nucleic acid in the above detection method and a sense strand of the wild-type genomic nucleic acid as the nucleic acid having a base sequence complementary to the probe nucleic acid, and by detecting nucleic acid mismatching between the probe nucleic acid, that is, the antisense strand of the wild-type genomic nucleic acid, and the sample nucleic acid in the same way as above, the mutation with respect to the wild-type genomic nucleic acid in the sample nucleic acid can be detected. In other words, another embodiment of the present invention provides a method for detecting mutation with respect to a standard nucleic acid in a sample nucleic acid, comprising the steps of:
(1) measuring a rise value between the melting temperatures of the standard nucleic acid and a nucleic acid having a base sequence complementary to the standard nucleic acid due to the presence of the specific amphoteric copolymer (standard ΔTm);
(2) measuring a rise value between the melting temperatures of the sample nucleic acid and the nucleic acid having a base sequence complementary to the standard nucleic acid due to the presence of the specific amphoteric copolymer (sample ΔTm); and
(3) determining the presence or absence of mutation based on a difference (ΔΔTm) between the sample ΔTm and the standard ΔTm.

### 4. Kit for Detecting Nucleic Acid Mismatching and Kit for Detecting Mutation

Another embodiment of the present invention provides kits for carrying out the above-described method for detecting nucleic acid mismatching between a sample nucleic acid and a probe nucleic acid and method for detecting mutation with respect to a standard nucleic acid in a sample nucleic acid. Specifically, one embodiment provides
a kit for detecting nucleic acid mismatching between a sample nucleic acid and a probe nucleic acid, comprising:
the probe nucleic acid;
a nucleic acid having a base sequence complementary to the probe nucleic acid; and
an agent for raising a melting temperature (Tm) of a double-stranded nucleic acid comprising a specific amphoteric copolymer.

Further, another embodiment provides
a kit for detecting mutation with respect to a standard nucleic acid in a sample nucleic acid, comprising:
the standard nucleic acid;
a nucleic acid having a base sequence complementary to the standard nucleic acid; and
an agent for raising a melting temperature (Tm) of a double-stranded nucleic acid comprising a specific amphoteric copolymer.

In these kits, details and preferred forms of the “probe nucleic acid”, “nucleic acid having a base sequence complementary to the probe nucleic acid”, “standard nucleic acid”, “nucleic acid having a base sequence complementary to the standard nucleic acid”, and “specific amphoteric copolymer” are as described above.

Further, it is also as described above that in addition to them, other components that can be used to measure a melting temperature (Tm) of a double-stranded nucleic acid, such as an aqueous medium, a buffer solution, a surfactant, a salt, other agents for raising a melting temperature (Tm), and a reagent for detecting a double-stranded nucleic acid may also be contained. In particular, from the point of facilitating the detection of nucleic acid mismatching and the detection of mutation, as the reagent for detecting a double-stranded nucleic acid, it is preferable to contain an intercalating fluorescent dye.

### EXAMPLES

Hereinafter, the present invention is further described by Examples, however, the present invention is not limited to the Examples.

### Example 1

A dimethylamino propyl methacrylamide hydrochloride/acrylamide/acrylic acid copolymer was evaluated for detection of mismatching by comparing a Tm rising range by a mismatch double strand with a Tm rising range by a full-match double strand with a polymer aqueous solution containing the copolymer as an agent for raising a Tm of a double-stranded nucleic acid.

### 1. Polymer Aqueous Solution (Agent for Raising Tm of Double-Stranded Nucleic Acid)

As an agent for raising a Tm of a double-stranded nucleic acid, a polymer aqueous solution having a pH of 7.0 and containing 10.0% by mass of a dimethylamino propyl methacrylamide hydrochloride/acrylamide/acrylic acid copolymer was prepared. The copolymer was obtained by copolymerizing a dimethylamino propyl methacrylamide hydrochloride (monomer 1), acrylamide (monomer 2), and acrylic acid (monomer 3) under the following polymerization condition 1 and has a molar ratio of a constituent unit 1 derived from monomer 1, a constituent unit 2 derived from monomer 2, and a constituent unit 3 derived from monomer 3 of 1:1:2.

Polymerization condition 1: Into a 300-mL four-neck flask equipped with a thermometer, a stirrer, and a cooling pipe, 16.15 g (equivalent to 0.04 mol) of a dimethylamino propyl methacrylamide hydrochloride (solid content concentration: 51.2% by mass), 2.93 g (equivalent to 0.04 mol) of acrylamide (solid content concentration: 97% by mass), 5.82 g (equivalent to 0.08 mol) of acrylic acid (solid content concentration: 99% by mass), and 143.86 g of distilled water were charged, and heated to 60° C. Every 2 hours, in the aqueous solution, 28.5% by mass of ammonium persulfate aqueous solution was added at such an amount that the amount of ammonium persulfate was 0.25, 0.5, 0.5, and 0.75 mol %, respectively to the whole amount of monomers, and the reaction was continued overnight.

### 2. Standard Nucleic Acid, Nucleic Acid Consisting of Complementary Sequence, and Nucleic Acid Having Mutation

In order to evaluate whether or not mutation at one base with respect to the nucleotide sequence of a standard nucleic acid is detectable, the following oligo DNAs were used.

TABLE 1

| | | |
|---|---|---|
| Reference nucleic acid | 11-mer oligo DNA derived from | GGTGGCGTAGG |
| | part of Exon 2 of wild-type KRAS | (SEQ ID NO: 1) |
| | gene sense strand | |
| Nucleic acid consisting of sequence | 11-mer oligo DNA of antisense | CCTACGCCACC |
| complementary to reference | strand consisting of sequence | (SEQ ID NO: 2) |
| nucleic acid (also referred to as | complementary to the sense | |
| complementary strand) | strand oligo DNA | |
| Nucleic acid having mutation with | 11-mer oligo DNA having | GGTGACGTAGG |
| respect to reference nucleic acid | mutation at one base with respect | (SEQ ID NO: 3) |
| (also referred to as mutation strand) | to the sense strand DNA oligomer | |

In a sequence of each oligo DNA in the table, a mismatch site is indicated with bold letter.

### 3. Preparation of Reaction Mixture

The following reaction mixtures 1 to 4 were prepared. A fluorescent dye in a fluorescent dye solution corresponds to an intercalating fluorescent dye.

TABLE 2

| | |
|---|---|
| Components | Content |

Reaction mixture 1

| | | |
|---|---|---|
| 500 μM of reference nucleic acid aqueous solution | 4.0 | μL |
| 500 μM of complementary strand aqueous solution | 4.0 | μL |
| 25 μM of fluorescent dye solution | 1.0 | μL |
| (manufactured by Biotium, trade name: EvaGreen) | | |
| Nuclease-free water | 11.0 | μL |

Reaction mixture 2

| | | |
|---|---|---|
| 500 μM of reference nucleic acid aqueous solution | 4.0 | μL |
| 500 μM of complementary strand aqueous solution | 4.0 | μL |
| 25 μM of fluorescent dye solution | 1.0 | μL |
| (manufactured by Biotium, trade name: EvaGreen) | | |
| 10.0% by mass of polymer aqueous solution | 2.0 | μL |
| Nuclease-free water | 9.0 | μL |

Reaction mixture 3

| | | |
|---|---|---|
| 500 μM of mutation strand aqueous solution | 4.0 | μL |
| 500 μM of complementary strand aqueous solution | 4.0 | μL |
| 25 μM of fluorescent dye solution | 1.0 | μL |
| (manufactured by Biotium, trade name: EvaGreen) | | |
| Nuclease-free water | 11.0 | μL |

Reaction mixture 4

| | | |
|---|---|---|
| 500 μM of mutation strand aqueous solution | 4.0 | μL |
| 500 μM of complementary strand aqueous solution | 4.0 | μL |
| 25 μM of fluorescent dye solution | 1.0 | μL |
| (manufactured by Biotium, trade name: EvaGreen) | | |
| 10.0% by mass of polymer aqueous solution | 2.0 | μL |
| Nuclease-free water | 9.0 | μL |

Concentration of the polymer (agent for raising Tm of a double-stranded nucleic acid) in reaction mixtures 2 and 4 was 1.0% by mass.

### 4. Creation of Double-Stranded Melting Curve

Each of the prepared reaction mixtures was set in StepOnePlus Real-Time PCR System (manufactured by Thermo Fisher Scientific), heated at 95° C. for 15 seconds, and then kept at 10° C. for 1 minute. Next, while performing fluorescence measurement every 0.3 seconds, the temperature was raised to 95° C. to obtain a double-stranded melting curve, and then held at 95° C. for 15 seconds.

### [Standard Example]

As a standard, a double-stranded melting curve was obtained with a magnesium chloride solution that is conventionally known as an agent for raising a Tm of a double-stranded nucleic acid, in place of the polymer aqueous solution.

Specifically, the following reaction mixtures 5 and 6 were prepared in place of the reaction mixtures 2 and 4, and a double-stranded melting curve was obtained from each reaction mixture in a similar manner to Example 1.

TABLE 3
Reaction mixture 5

| Components | Content | |
|---|---|---|
| 500 μM of reference nucleic acid aqueous solution | 4.0 | μL |
| 500 μM of complementary strand aqueous solution | 4.0 | μL |
| 25 μM of fluorescent dye solution | 1.0 | μL |
| (manufactured by Biotium, trade name: EvaGreen) | | |
| 15 mM MgCl₂ aqueous solution | 2.0 | μL |
| Nuclease-free water | 9.0 | μL |

Reaction mixture 6

| [symbol] | Content | |
|---|---|---|
| 500 μM of mutation strand aqueous solution | 4.0 | μL |
| 500 μM of complementary strand aqueous solution | 4.0 | μL |
| 25 μM of fluorescent dye solution | 1.0 | μL |
| (manufactured by Biotium, trade name: EvaGreen) | | |
| 15 mM MgCl₂ aqueous solution | 2.0 | μL |
| Nuclease-free water | 9.0 | μL |

### [Test Results]

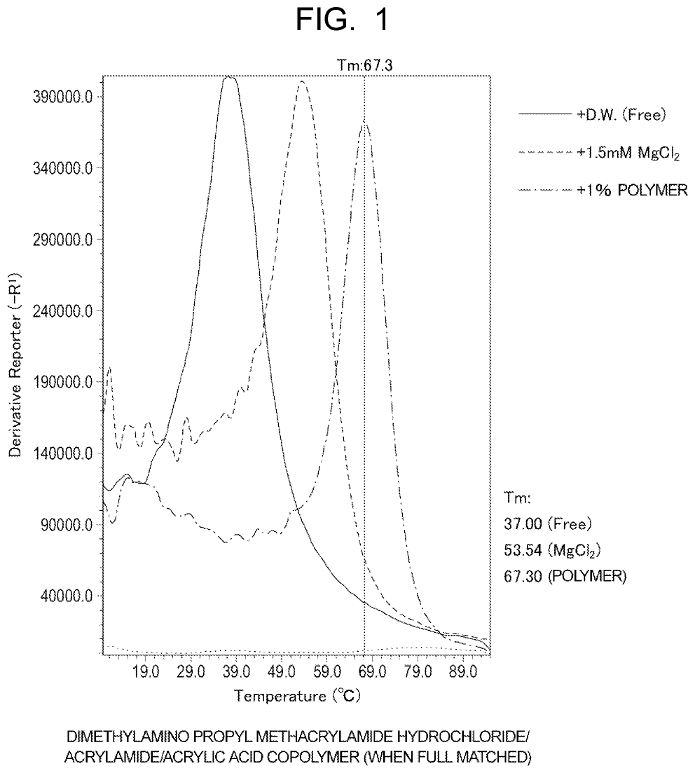

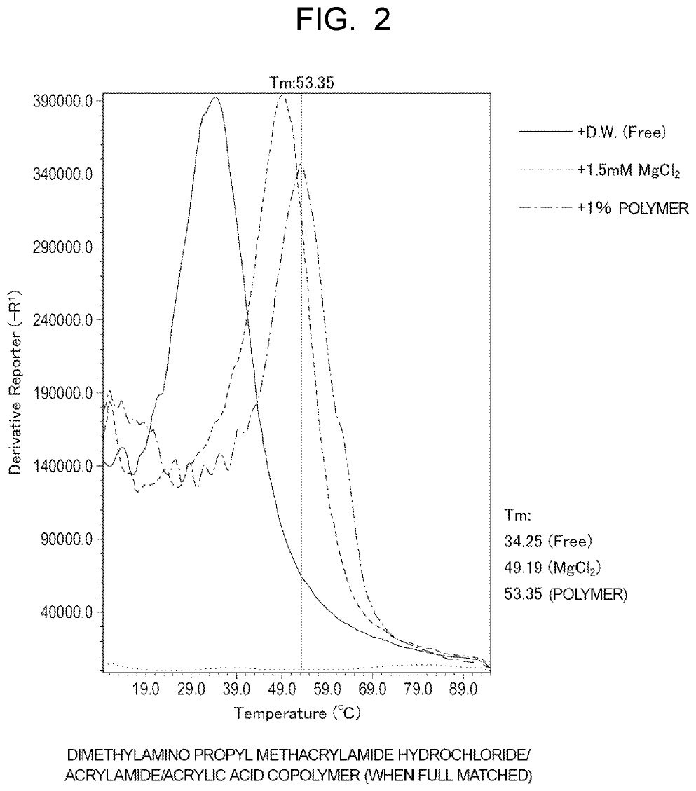

Double-stranded melting curves obtained from the reaction mixtures 1, 2 and 5 are shown in FIG. 1, and double-stranded melting curves obtained from the reaction mixtures 3, 4 and 6 are shown in FIG. 2. As shown in FIG. 1, a Tm value of a double-strand between the standard nucleic acid and the complementary strand was 37.0° C. in the absence of the polymer (agent for raising Tm of a double-stranded nucleic acid). On the other hand, a Tm value of a double-strand between the standard nucleic acid and the complementary strand was 67.30° C. in the presence of the polymer (agent for raising Tm of a double-stranded nucleic acid), and a 30.30° C.-rise in the Tm value was observed.

Further, as shown in FIG. 2, a Tm value of a double-strand between the mutation strand and the complementary strand was 34.25° C. in the absence of the polymer (agent for raising Tm of a double-stranded nucleic acid). On the other hand, a Tm value of a double-strand between the mutation strand and the complementary strand was 53.35° C. in the presence of the polymer (agent for raising Tm of a double-stranded nucleic acid), and a 19.10° C.-rise in the Tm value was observed.

As a result, a difference of 11.20° C. was observed between the rise in the Tm value of a double-strand between the standard nucleic acid and the complementary strand and the rise in the Tm value of a double-strand between the mutation strand and the complementary strand in the presence of the polymer (agent for raising Tm of a double-stranded nucleic acid).

### Example 2

A melting curve of a double strand was obtained from each reaction mixture in a similar manner to Example 1 except that a diallylamine hydrochloride/maleic acid copolymer obtained by copolymerizing a diallylamine hydrochloride (monomer 1) and maleic acid (monomer 2) under the following polymerization condition 2 and having a molar ratio of a constituent unit 1 derived from the monomer 1 and a constituent unit 2 derived from the monomer 2 of 1:1 was used as the agent for raising a Tm of a double-stranded nucleic acid, and then the Tm rising range (standard ΔTm) between a standard nucleic acid and a complementary strand and Tm rising range (sample ΔTm) between a mutation strand and a complementary strand due to the presence of a polymer (agent for raising a Tm of a double-stranded nucleic acid) were determined, and the difference (ΔΔTm) between them was calculated.

Polymerization condition 2: Into a 500-mL four-neck flask equipped with a thermometer, a stirrer, and a cooling pipe, 160.07 g (0.80 mol) of 66.78% diallylamine hydrochloride, 78.45 g (0.80 mol) of maleic anhydride, and 91.86 g of distilled water were charged, and the internal temperature was raised to 50° C. 28.5% by mass of ammonium persulfate aqueous solution was added at such an amount that the amount of ammonium persulfate in the aqueous solution was 0.5% by mass to the whole amount of monomers, and the polymerization was started. The ammonium persulfate aqueous solution was added at such an amount that the amount of ammonium persulfate was 0.5% by mass after 4 hours, 1.0% by mass after 20 and 26 hours, and 1.5% by mass after 45 and 51 hours to the whole amount of monomers, and the mixture was reacted for 68 hours.

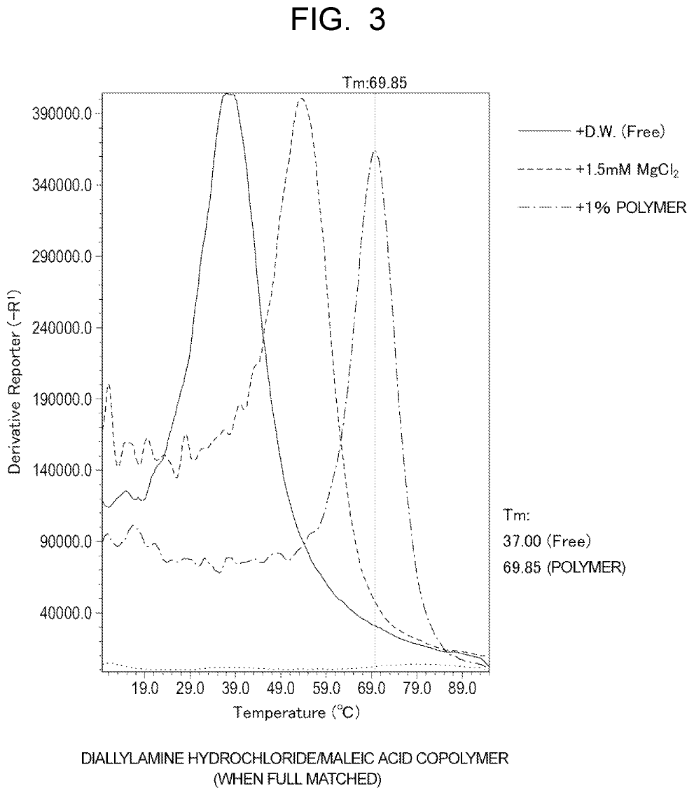

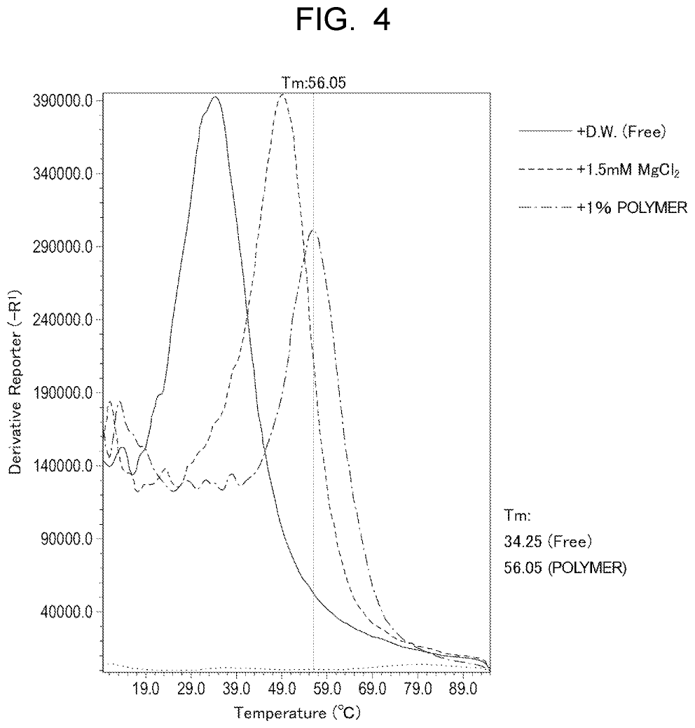

FIG. 3 shows melting curves of a double strand obtained from the reaction mixtures 1, 2 and 5, and FIG. 4 shows melting curves of a double strand obtained from the reaction mixtures 3, 4 and 6. As shown in FIG. 3, a Tm value of a double strand between the standard nucleic acid and the complementary strand was 37.00° C. in the absence of the polymer (agent for raising a Tm of a double-stranded nucleic acid). On the other hand, a Tm value of a double strand between the standard nucleic acid and the complementary strand was 69.85° C. in the presence of the polymer (agent for raising a Tm of a double-stranded nucleic acid), and a 32.85° C.-rise in the Tm value was observed.

Further, as shown in FIG. 4, a Tm value of a double strand between the mutation strand and the complementary strand was 34.25° C. in the absence of the polymer (agent for raising a Tm of a double-stranded nucleic acid). On the other hand, a Tm value of a double strand between the mutation strand and the complementary strand was 56.05° C. in the presence of the polymer (agent for raising a Tm of a double-stranded nucleic acid), and a 21.80-rise in the Tm value was observed.

As a result, a difference of 11.05° C. was observed between the rise in the Tm value of a double strand between the standard nucleic acid and the complementary strand and the rise in the Tm value of a double strand between the mutation strand and the complementary strand in the presence of the polymer (agent for raising a Tm of a double-stranded nucleic acid).

### Example 3

A melting curve of a double strand was obtained from each reaction mixture in a similar manner to Example 1 except that a diallylmethylamine/maleic acid copolymer obtained by copolymerizing diallylmethylamine (monomer 1) and maleic acid (monomer 2) under the following polymerization condition 3 and having a molar ratio of a constituent unit 1 derived from the monomer 1 and a constituent unit 2 derived from the monomer 2 of 1:1 was used as the agent for raising a Tm of a double-stranded nucleic acid, and then the Tm rising range (standard ΔTm) between the standard nucleic acid and the complementary strand and Tm rising range (sample ΔTm) between the mutation strand and the complementary strand due to the presence of a polymer (agent for raising a Tm of a double-stranded nucleic acid) were determined, and the difference (ΔΔTm) between them was calculated.

Polymerization condition 3: Into a 20-L four-neck flask equipped with a thermometer, a stirrer, and a cooling pipe, 1.86 kg (19.0 mol) of maleic anhydride and 0.34 kg of distilled water were charged, and 2.11 kg (19.0 mol) of diallylmethylamine (DAMA) was added dropwise under cooling. After that, the internal temperature was raised to 50° C. 28.5% by mass of ammonium persulfate aqueous solution was added at such an amount that the amount of ammonium persulfate in the aqueous solution was 0.5% by mass to the whole amount of monomers, and the polymerization was started. After 3, 21, and 25 hours, the 28.5% by mass of ammonium persulfate aqueous solution was added at such an amount that the amount of ammonium persulfate in the aqueous solution was 1.0% by mass to the whole amount of monomers, and the mixture was further reacted overnight.

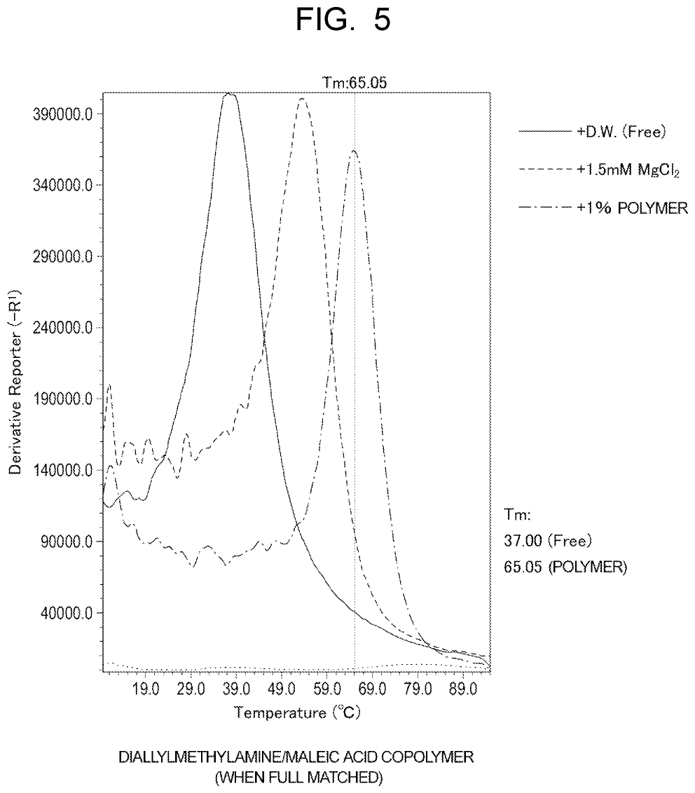

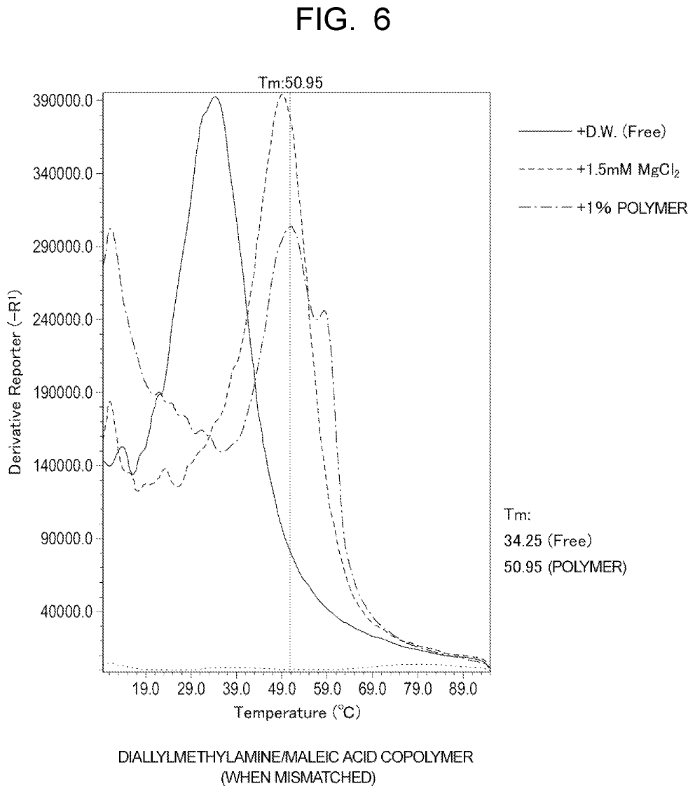

FIG. 5 shows melting curves of a double strand obtained from the reaction mixtures 1, 2 and 5, and FIG. 6 shows melting curves of a double strand obtained from the reaction mixtures 3, 4 and 6. As shown in FIG. 5, a Tm value of a double strand between the standard nucleic acid and the complementary strand was 37.00° C. in the absence of the polymer (agent for raising a Tm of a double-stranded nucleic acid). On the other hand, a Tm value of a double strand between the standard nucleic acid and the complementary strand was 65.05° C. in the presence of the polymer (agent for raising a Tm of a double-stranded nucleic acid), and a 28.05° C.-rise in the Tm value was observed.

Further, as shown in FIG. 6, a Tm value of a double strand between the mutation strand and the complementary strand was 34.25° C. in the absence of the polymer (agent for raising a Tm of a double-stranded nucleic acid). On the other hand, a Tm value of a double strand between the mutation strand and the complementary strand was 50.95° C. in the presence of the polymer (agent for raising a Tm of a double-stranded nucleic acid), and a 16.70-rise in the Tm value was observed.

As a result, a difference of 11.35° C. was observed between the rise in the Tm value of a double strand between the standard nucleic acid and the complementary strand and the rise in the Tm value of a double strand between the mutation strand and the complementary strand in the presence of the polymer (agent for raising a Tm of a double-stranded nucleic acid).

### Example 4

An oligo-oligo double strand melting test was performed except that a diallylamine hydrochloride/acrylamide/acrylic acid copolymer obtained by copolymerizing a diallylamine hydrochloride (monomer 1), acrylamide (monomer 2), and acrylic acid (monomer 3) under the following polymerization condition 4 and having a molar ratio of a constituent unit 1 derived from the monomer 1, a constituent unit 2 derived from the monomer 2, and a constituent unit 3 derived from the monomer 3 of 1:1:2 was used as the agent for raising a Tm of a double-stranded nucleic acid.

Polymerization condition 4: Into a 300-mL four-neck flask equipped with a thermometer, a stirrer, and a cooling pipe, 20.43 g (equivalent to 0.1 mol) of diallylamine hydrochloride (solid content concentration: 65.40% by mass) and 110.9 g of distilled water were charged, and heated to 65° C. 28.5% by mass of ammonium persulfate aqueous solution was added at such an amount that the amount of ammonium persulfate in an aqueous solution was 2.0 mol % to the whole amount of monomers, and after the lapse of 30 minutes, a solution obtained by mixing 7.11 g (equivalent to 0.1 mol) of acrylamide (solid content concentration: 97% by mass), 14.56 g (equivalent to 0.2 mol) of acrylic acid (solid content concentration: 99% by mass), and 21.15 g of distilled water was added dropwise over 3 hours, and the reaction was continued overnight.

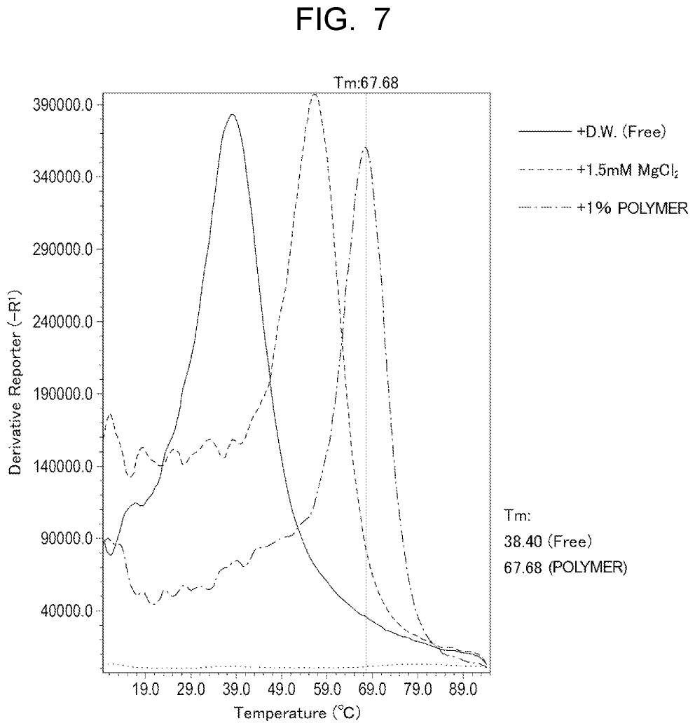

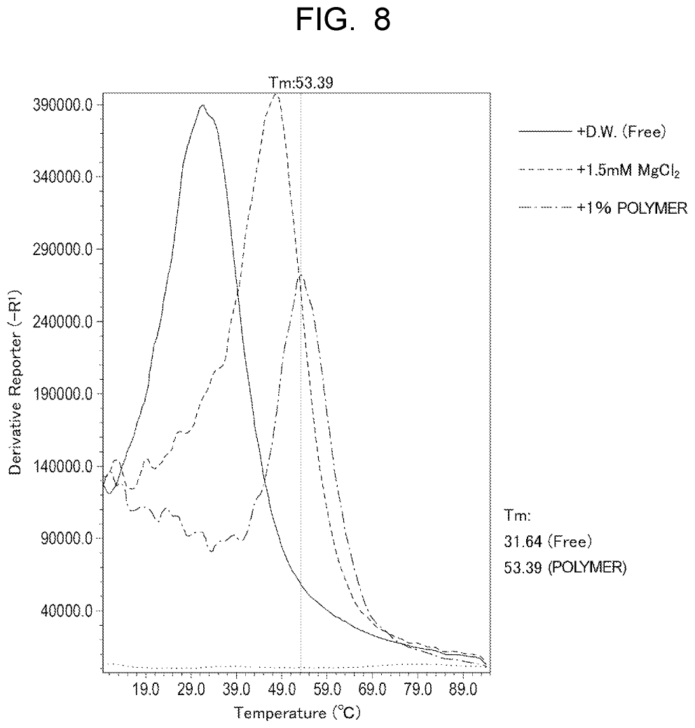

FIG. 7 shows melting curves of a double strand obtained from the reaction mixtures 1, 2 and 5, and FIG. 8 shows melting curves of a double strand obtained from the reaction mixtures 3, 4 and 6. As shown in FIG. 7, a Tm value of a double strand between the standard nucleic acid and the complementary strand was 38.40° C. in the absence of the polymer (agent for raising a Tm of a double-stranded nucleic acid). On the other hand, a Tm value of a double strand between the standard nucleic acid and the complementary strand was 67.68° C. in the presence of the polymer (agent for raising a Tm of a double-stranded nucleic acid), and a 29.28° C.-rise in the Tm value was observed.

Further, as shown in FIG. 8, a Tm value of a double strand between the mutation strand and the complementary strand was 31.64° C. in the absence of the polymer (agent for raising a Tm of a double-stranded nucleic acid). On the other hand, a Tm value of a double strand between the mutation strand and the complementary strand was 53.39° C. in the presence of the polymer (agent for raising a Tm of a double-stranded nucleic acid), and a 21.75-rise in the Tm value was observed.

As a result, a difference of 7.53° C. was observed between the rise in the Tm value of a double strand between the standard nucleic acid and the complementary strand and the rise in the Tm value of a double strand between the mutation strand and the complementary strand in the presence of the polymer (agent for raising a Tm of a double-stranded nucleic acid).

### Comparative Example 1

A melting curve of a double strand was obtained from each reaction mixture in a similar manner to Example 1 except that an allylamine polymer (product name: PAA-03, manufactured by Nittobo Medical Co., Ltd.) having a weight average molecular weight (Mw) of 3000 obtained by GPC measurement was used as the agent for raising a Tm of a double-stranded nucleic acid, and then the Tm rising range (standard ΔTm) between the standard nucleic acid and the complementary strand and Tm rising range (sample ΔTm) between the mutation strand and the complementary strand due to the presence of a polymer (agent for raising a Tm of a double-stranded nucleic acid) were determined, and the difference (ΔΔTm) between them was calculated.

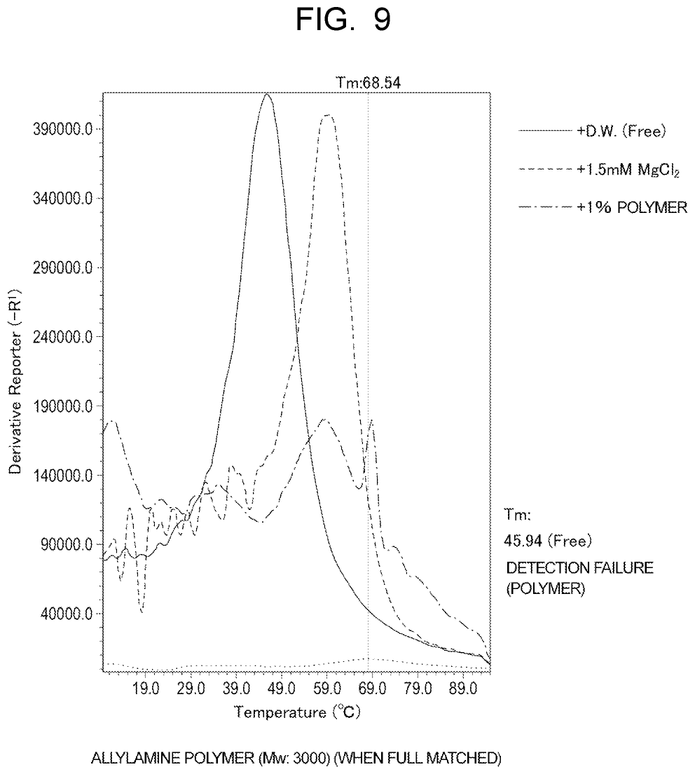

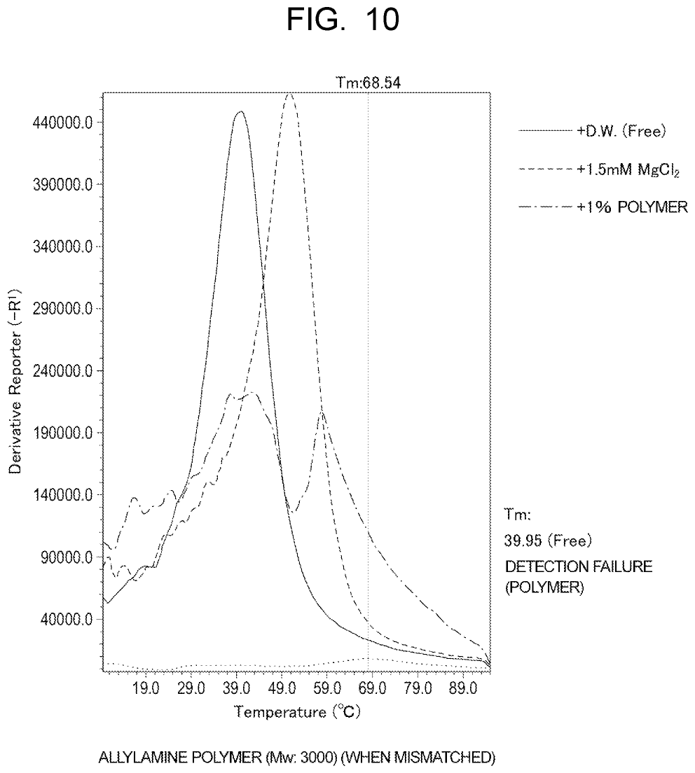

FIG. 9 shows melting curves of a double strand obtained from the reaction mixtures 1, 2 and 5, and FIG. 10 shows melting curves of a double strand obtained from the reaction mixtures 3, 4 and 6. As shown in FIGS. 9 and 10, in the presence of the polymer (agent for raising a Tm of a double-stranded nucleic acid), no clear peak was detected, and it was difficult to determine the Tm value.

### Comparative Example 2

A melting curve of a double strand was obtained from each reaction mixture in a similar manner to Example 1 except that an allylamine polymer (product name: PAA-08, manufactured by Nittobo Medical Co., Ltd.) having a weight average molecular weight (Mw) of 8000 obtained by GPC measurement was used as the agent for raising a Tm of a double-stranded nucleic acid, and then the Tm rising range (standard ΔTm) between the standard nucleic acid and the complementary strand and Tm rising range (sample ΔTm) between the mutation strand and the complementary strand due to the presence of a polymer (agent for raising a Tm of a double-stranded nucleic acid) were determined, and the difference (ΔΔTm) between them was calculated.

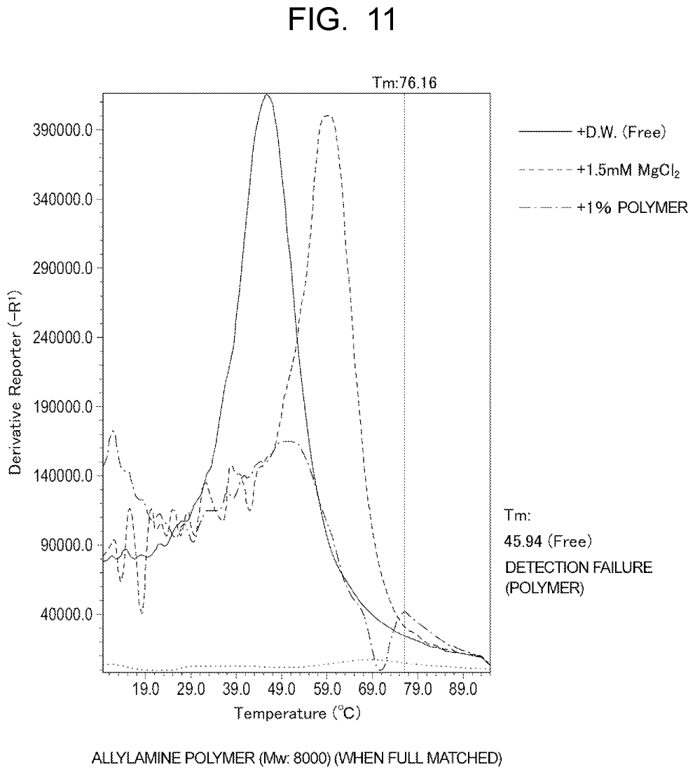

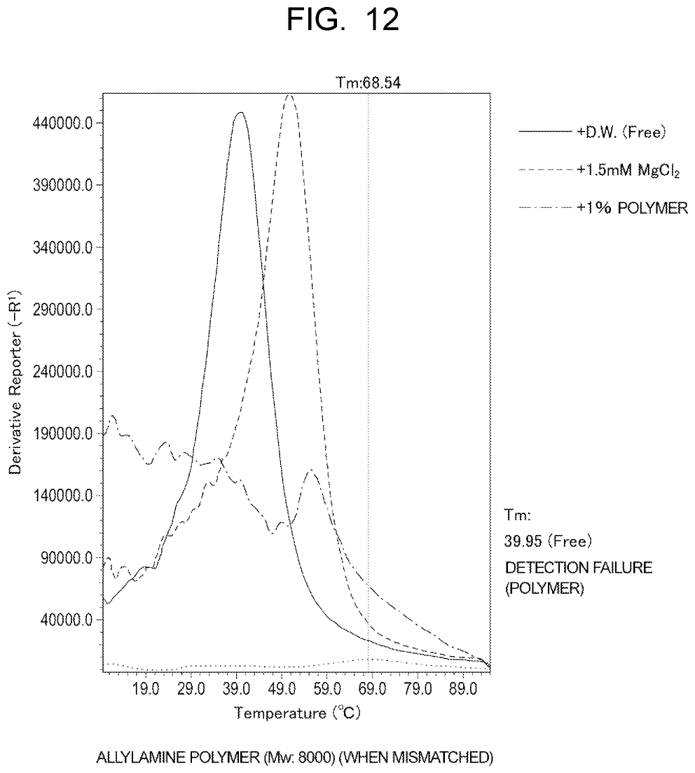

FIG. 11 shows melting curves of a double strand obtained from the reaction mixtures 1, 2 and 5, and FIG. 12 shows melting curves of a double strand obtained from the reaction mixtures 3, 4 and 6. As shown in FIGS. 11 and 12, in the presence of the polymer (agent for raising a Tm of a double-stranded nucleic acid), no clear peak was detected, and it was difficult to determine the Tm value.

### Comparative Example 3

A melting curve of a double strand was obtained from each reaction mixture in a similar manner to Example 1 except that an allylamine polymer (product name: PAA-15C, manufactured by Nittobo Medical Co., Ltd.) having a weight average molecular weight (Mw) of 15000 obtained by GPC measurement was used as the agent for raising a Tm of a double-stranded nucleic acid, and then the Tm rising range (standard ΔTm) between the standard nucleic acid and the complementary strand and Tm rising range (sample ΔTm) between the mutation strand and the complementary strand due to the presence of a polymer (agent for raising a Tm of a double-stranded nucleic acid) were determined, and the difference (ΔΔTm) between them was calculated.

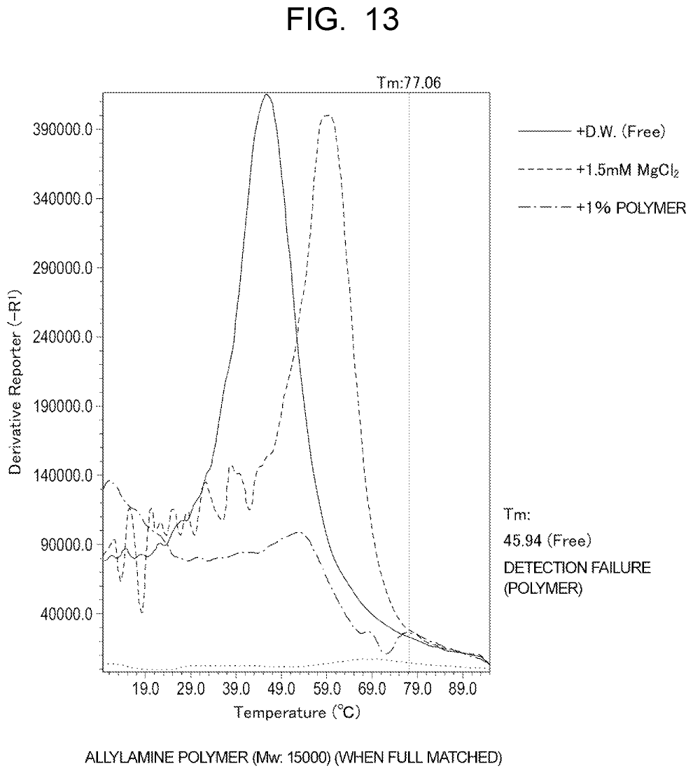

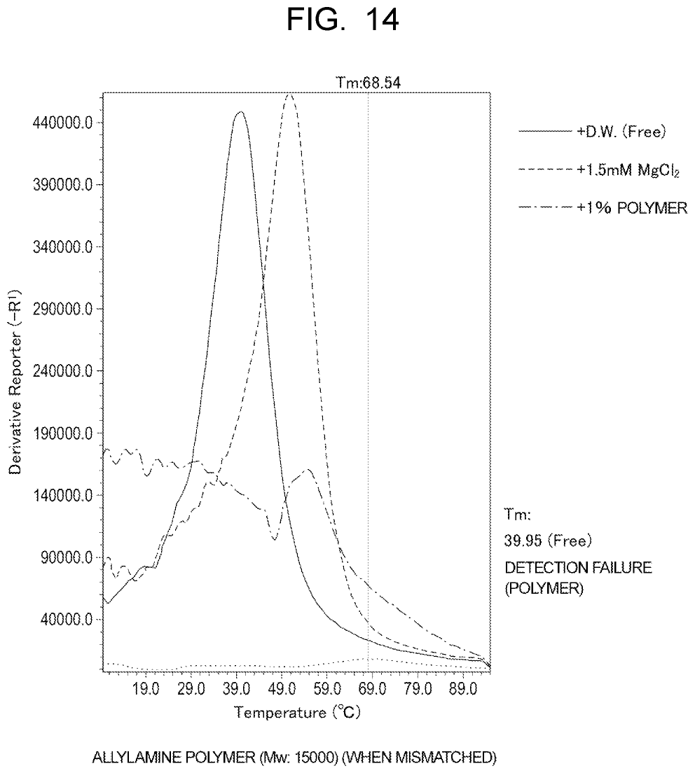

FIG. 13 shows melting curves of a double-strand obtained from the reaction mixtures 1, 2 and 5, and FIG. 14 shows melting curves of a double strand obtained from the reaction mixtures 3, 4 and 6. As shown in FIGS. 13 and 14, in the presence of the polymer (agent for raising a Tm of a double-stranded nucleic acid), no clear peak was detected, and it was difficult to determine the Tm value.

### Comparative Example 4

An oligo-oligo double strand melting test was performed except that an allylamine hydrochloride/diallylamine hydrochloride copolymer obtained by copolymerizing an allylamine hydrochloride (monomer 1) and a diallylamine hydrochloride (monomer 2) under the following polymerization condition 5 and having a molar ratio of a constituent unit 1 derived from the monomer 1 and a constituent unit 2 derived from the monomer 2 of 1:1 and a weight average molecular weight (Mw) of 100000 obtained by GPC measurement was used as the agent for raising a Tm of a double-stranded nucleic acid.

Polymerization condition 5: Into a 1-L four-neck flask equipped with a thermometer, a stirrer, and a cooling pipe, 245.24 g (1.50 mol) of 57.22% by mass of allylamine hydrochloride and 307.31 g (1.50 mol) of 65.22% by mass of diallylamine hydrochloride were charged, and heated to 60° C. 30% by mass of V-50 (2,2′-azobis (2-methylpropionamidine) dihydrochloride) suspension was added at such an amount that the amount of V-50 in the suspension was 0.50% by mass to the whole amount of monomers, and the polymerization was started. After 3 hours, 30% by mass of V-50 (2,2′-azobis(2-methylpropionamidine)dihydrochloride) suspension was added at such an amount that the amount of V-50 in the suspension was 0.50% by mass, and after 24, 27, 48, 51, and 54 hours, at such an amount that the amount of V-50 in the suspension was 0.25% by mass, to the whole amount of monomers were added, and the mixture was further reacted overnight.

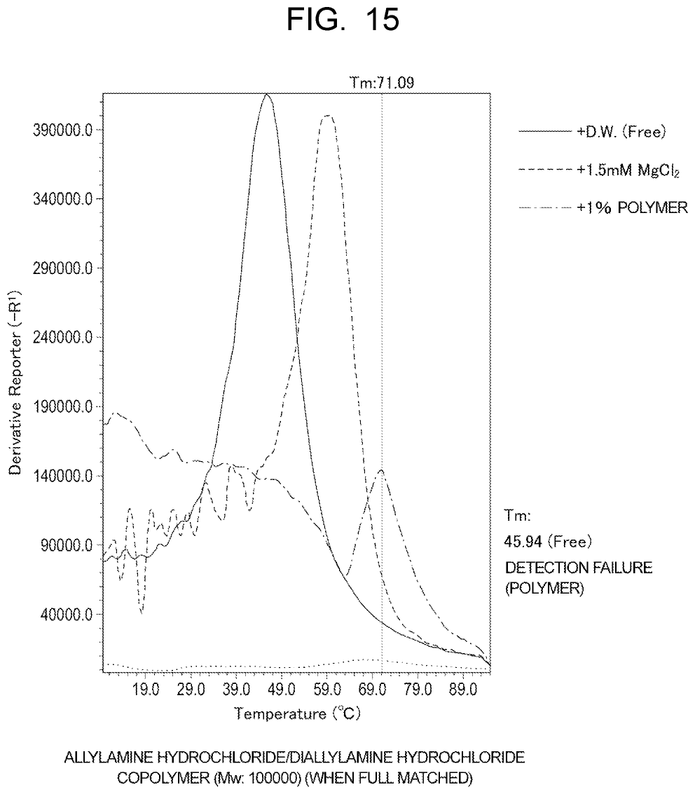

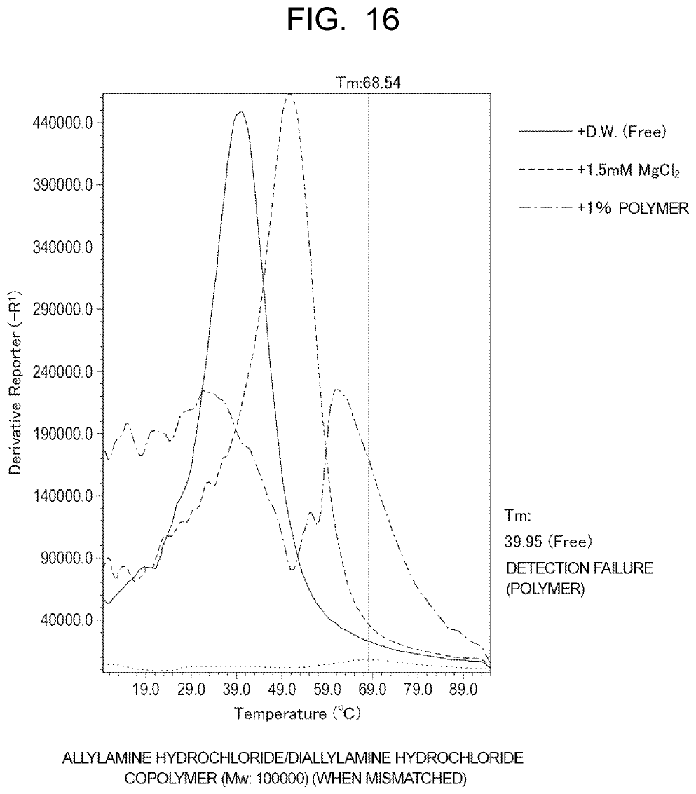

FIG. 15 shows melting curves of a double strand obtained from the reaction mixtures 1, 2 and 5, and FIG. 16 shows melting curves of a double strand obtained from the reaction mixtures 3, 4 and 6. As shown in FIGS. 15 and 16, in the presence of the polymer (agent for raising a Tm of a double-stranded nucleic acid), no clear peak was detected, and it was difficult to determine the Tm value.

### Comparative Example 5

An oligo-oligo double strand melting test was performed except that an allylamine hydrochloride/diallylamine hydrochloride copolymer obtained by copolymerizing an allylamine hydrochloride (monomer 1) and a diallylamine hydrochloride (monomer 2) under the following polymerization condition 6 and having a molar ratio of a constituent unit 1 derived from the monomer 1 and a constituent unit 2 derived from the monomer 2 of 4:1 and a weight average molecular weight (Mw) of 20000 obtained by GPC measurement was used as the agent for raising a Tm of a double-stranded nucleic acid.

Polymerization condition 6: Into a 1-L four-neck flask equipped with a thermometer, a stirrer, and a cooling pipe, 490.48 g (3.00 mol) of 57.22% by mass of allylamine hydrochloride and 153.66 g (0.75 mol) of 65.22% by mass of diallylamine hydrochloride were charged, and heated to 60° C. 30% by mass of V-50 (2,2′-azobis(2-methylpropionamidine)dihydrochloride) suspension was added at such an amount that the amount of V-50 in the suspension was 1.00% by mass to the whole amount of monomers, and the polymerization was started. After 24 hours, 30% by mass of V-50 (2,2′-azobis(2-methylpropionamidine)dihydrochloride) suspension was added at such an amount that the amount of V-50 in the suspension was 1.00% by mass, and after 48 hours, at such an amount that the amount of V-50 in the suspension was 0.50% by mass were added, and the mixture was further reacted overnight.

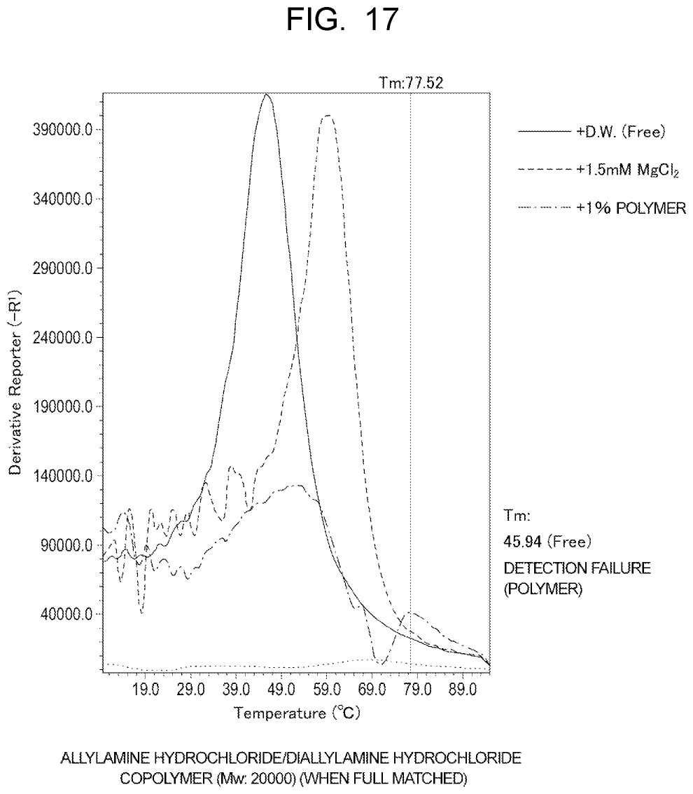

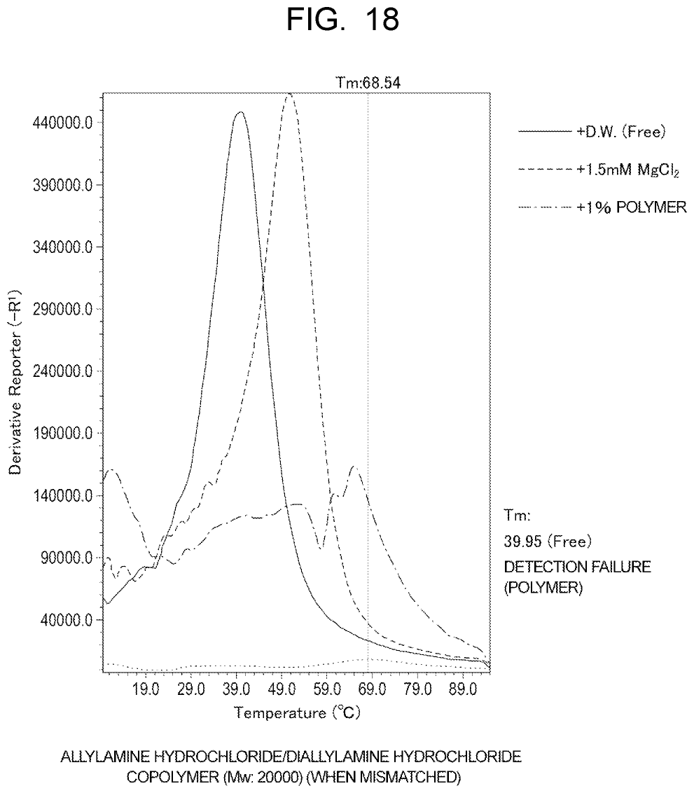

FIG. 17 shows melting curves of a double strand obtained from the reaction mixtures 1, 2 and 5, and FIG. 18 shows melting curves of a double strand obtained from the reaction mixtures 3, 4 and 6. As shown in FIGS. 17 and 18, in the presence of the polymer (agent for raising a Tm of a double-stranded nucleic acid), no clear peak was detected, and it was difficult to determine the Tm value.

### Comparative Example 6

A melting curve of a double strand was obtained from each reaction mixture in a similar manner to Example 1 except that 50 mol % acetylated polyallylamine having a weight average molecular weight (Mw) of 15000 obtained by GPC measurement obtained under the following reaction condition 1 was used, and then the Tm rising range (standard ΔTm) between the standard nucleic acid and the complementary strand and Tm rising range (sample ΔTm) between the mutation strand and the complementary strand due to the presence of a polymer (agent for raising a Tm of a double-stranded nucleic acid) were determined, and the difference (ΔΔTm) between them was calculated.

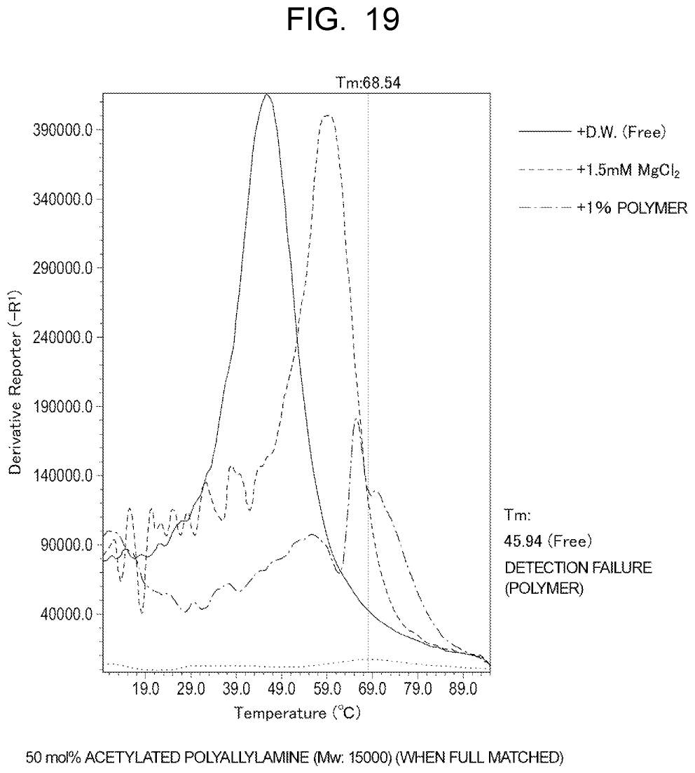

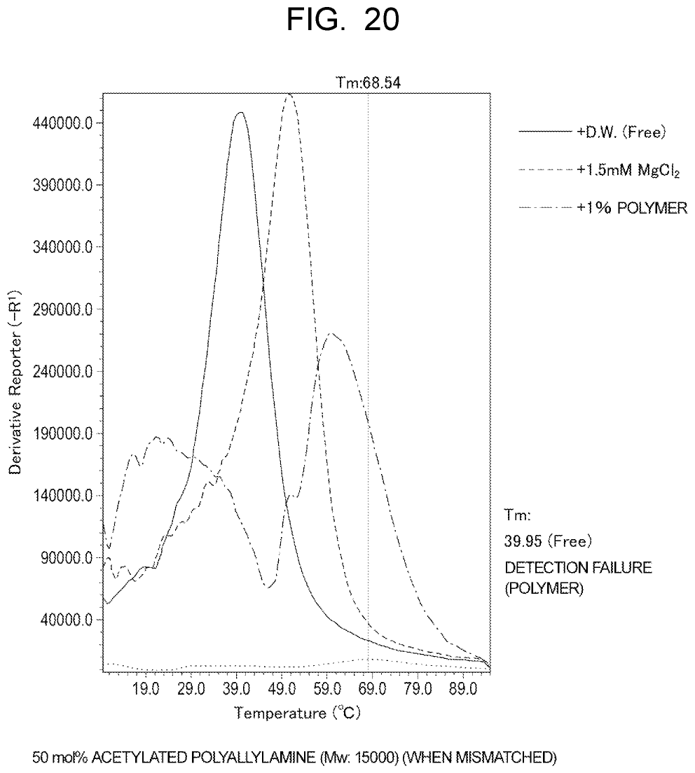

FIG. 19 shows melting curves of a double strand obtained from the reaction mixtures 1, 2 and 5, and FIG. 20 shows melting curves of a double strand obtained from the reaction mixtures 3, 4 and 6. As shown in FIGS. 19 and 20, in the presence of the polymer (agent for raising a Tm of a double-stranded nucleic acid), no clear peak was detected, and it was difficult to determine the Tm value.

Reaction condition 1: Into a 20-L four-neck flask equipped with a thermometer, a stirrer, and a cooling pipe, 17.00 kg (45.11 mol) of 15.15% by mass of allylamine polymer (product name: PAA-15C, manufactured by Nittobo Medical Co., Ltd.) was charged. After that, 2.37 kg (22.55 mol) of acetic anhydride was added dropwise while cooling, and the mixture was reacted overnight at room temperature.

### Comparative Example 7

A melting curve of a double strand was obtained from each reaction mixture in a similar manner to Example 1 except that polyacrylamide having a weight average molecular weight (Mw) of 20000 obtained by GPC measurement obtained by polymerization under the following polymerization condition 7 was used, and then the Tm rising range (standard ΔTm) between the standard nucleic acid and the complementary strand and Tm rising range (sample ΔTm) between the mutation strand and the complementary strand due to the presence of a polymer (agent for raising a Tm of a double-stranded nucleic acid) were determined, and the difference (ΔΔTm) between them was calculated.

Polymerization condition 7: Into a 300-mL four-neck flask equipped with a thermometer, a stirrer, and a cooling pipe, 186.59 g of distilled water was charged, and heated to 60° C. 0.75 g of ammonium persulfate was added, and then a solution obtained by mixing 25.65 g (equivalent to 0.35 mol) of acrylamide (solid content concentration: 97% by mass) and 36.55 g of distilled water was added dropwise over 5 hours, and the reaction was continued overnight.

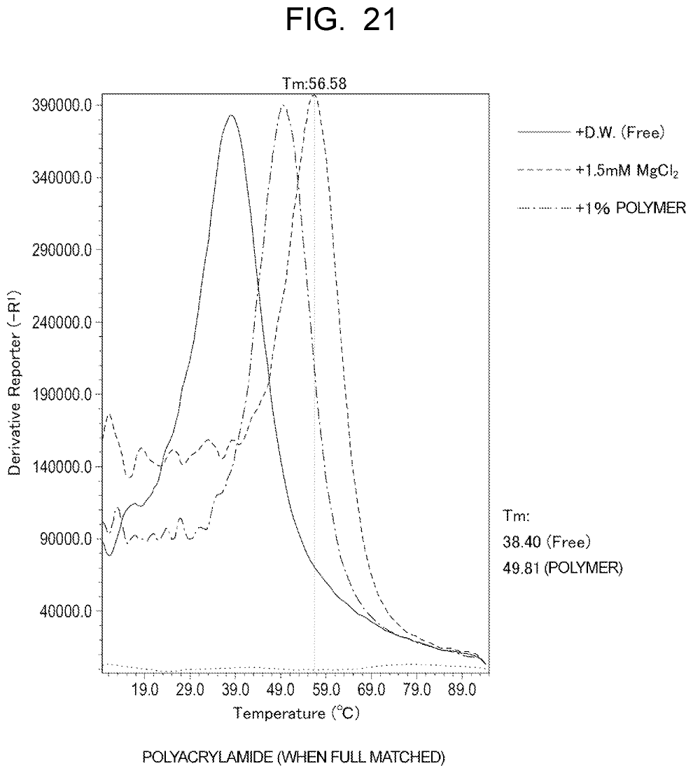

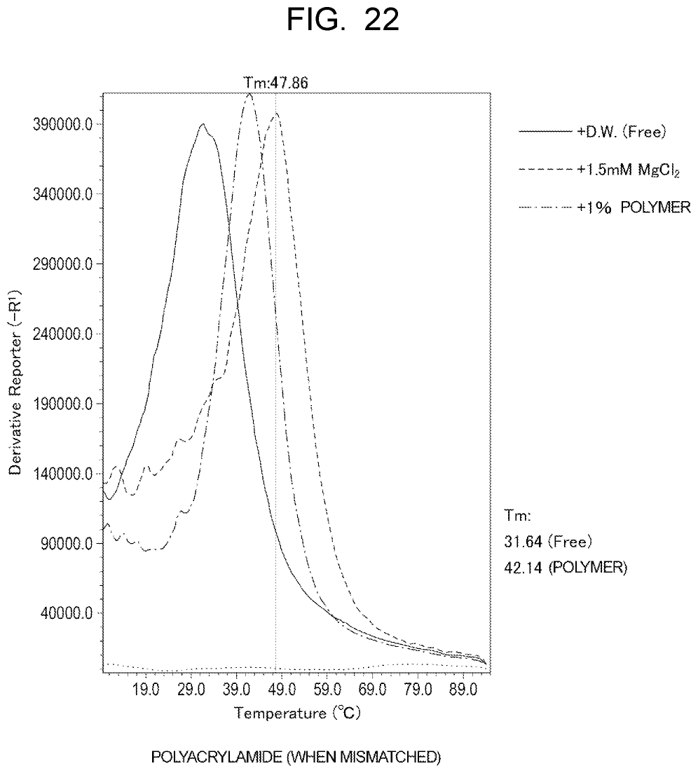

FIG. 21 shows melting curves of a double strand obtained from the reaction mixtures 1, 2 and 5, and FIG. 22 shows melting curves of a double strand obtained from the reaction mixtures 3, 4 and 6. As shown in FIG. 21, a Tm value of a double strand between the standard nucleic acid and the complementary strand was 38.4° C. in the absence of the polymer (agent for raising a Tm of a double-stranded nucleic acid). On the other hand, a Tm value of a double strand between the standard nucleic acid and the complementary strand was 49.81° C. in the presence of the polymer (agent for raising a Tm of a double-stranded nucleic acid), and a 11.41° C.-rise in the Tm value was observed.

Further, as shown in FIG. 22, a Tm value of a double strand between the mutation strand and the complementary strand was 31.64° C. in the absence of the polymer (agent for raising a Tm of a double-stranded nucleic acid). On the other hand, a Tm value of a double strand between the mutation strand and the complementary strand was 42.14° C. in the presence of the polymer (agent for raising a Tm of a double-stranded nucleic acid), and a 10.50-rise in the Tm value was observed.

As a result, only a difference of 0.91° C. was observed between the rise in the Tm value of a double strand between the standard nucleic acid and the complementary strand and the rise in the Tm value of a double strand between the mutation strand and the complementary strand in the presence of the polymer (agent for raising a Tm of a double-stranded nucleic acid).

The test results are summarized below.

TABLE 4

| | Example 1 | Example 2 | Example 3 | Example 4 |
|---|---|---|---|---|
| Agent for | Dimethylamino | Diallylamine | Diallylmethylamine/ | Diallylamine |
| raising Tm of | propyl methacrylamide | hydrochloride/ | maleic acid | hydrochloride/ |
| double-stranded | hydrochloride/ | maleic acid | copolymer | acrylamide/ |
| nucleic acid | acrylamide/ | copolymer | | acrylic acid |
| | acrylic acid | | | copolymer |
| | copolymer | | | |
| Molar ratio of | | | | |
| constituent unit | | | | |
| Cationic unit | 1 | 1 | 1 | 1 |
| Anionic unit | 2 | 1 | 1 | 2 |
| Nonionic unit | 1 | 0 | 0 | 1 |
| Cation/anion | 0.5 | 1 | 1 | 0.5 |
| Reference ΔTm | 30.30 | 32.85 | 28.05 | 29.28 |
| (° C.) | | | | |
| Sample ΔTm | 19.10 | 21.80 | 16.70 | 21.75 |
| (° C.) | | | | |
| Δ ΔTm (° C.) | 11.20 | 11.05 | 11.35 | 7.53 |

TABLE 5

| | Comparative | Comparative | Comparative | Comparative |
|---|---|---|---|---|
| | Example 1 | Example 2 | Example 3 | Example 4 |
| Agent for | Polyallylamine | Polyallylamine | Polyallylamine | Allylamine |
| raising Tm of | (Mw: 3000) | (Mw: 8000) | (Mw: 15000) | hydrochloride/ |
| double-stranded | | | | diallylamine |
| nucleic acid | | | | hydrochloride |
| | | | | copolymer |
| | | | | (Mw: 100000) |
| Molar ratio of | | | | |
| constituent unit | | | | |
| Cationic unit | 1 | 1 | 1 | 1 |
| Anionic unit | 0 | 0 | 0 | 0 |
| Nonionic unit | 0 | 0 | 0 | 0 |
| Cation/anion | — | — | — | — |
| Reference ΔTm | (Detection | (Detection | (Detection | (Detection |
| (° C.) | failure) | failure) | failure) | failure) |
| Sample ΔTm | (Detection | (Detection | (Detection | (Detection |
| (° C.) | failure) | failure) | failure) | failure) |
| Δ ΔTm (° C.) | — | — | — | — |

TABLE 6

| | Comparative | Comparative | Comparative | Reference |
|---|---|---|---|---|
| | Example 5 | Example 6 | Example 7 | Example 1 |
| Agent for | Allylamine | 50 mol% | Polyacrylamide | Magnesium |
| raising Tm of | hydrochloride/ | Acetylated | (Mw: 20000) | chloride |
| double-stranded | diallylamine | polyallylamine | | |
| nucleic acid | hydrochloride | (Mw: 15000) | | |
| | copolymer | | | |
| | (Mw: 20000) | | | |
| Molar ratio of | | | | — |
| constituent unit | | | | |
| Cationic unit | 1 | 1 | 0 | — |
| Anionic unit | 0 | 0 | 0 | — |
| Nonionic unit | 0 | 1 | 1 | — |
| Cation/anion | — | — | — | 1 |
| Reference ΔTm | (Detection | (Detection | 11.4 | 16.54 |
| (° C.) | failure) | failure) | | |
| Sample ΔTm | (Detection | (Detection | 10.5 | 14.94 |
| (° C.) | failure) | failure) | | |
| Δ ΔTm (° C.) | — | — | 0.9 | 1.6 |

### Example 5

By using artificial oligo DNA that was partly constituted of BNA as the complementary strand with respect to a standard nucleic acid, a dimethylamino propyl methacrylamide hydrochloride/acrylamide/acrylic acid copolymer was evaluated for the detection of mismatching in a similar manner to Example 1.

### 1. Polymer Aqueous Solution (Agent for Raising Tm of Double-Stranded Nucleic Acid)

As the agent for raising a Tm of a double-stranded nucleic acid, a polymer aqueous solution containing 10.0% by mass of the dimethylamino propyl methacrylamide hydrochloride/acrylamide/acrylic acid copolymer used in Example 1 and having a pH of 7.0 was prepared.

### 2. Standard Nucleic Acid, Nucleic Acid Consisting of Complementary Sequence, and Nucleic Acid Having Mutation

In order to evaluate whether or not mutation at one base with respect to the nucleotide sequence of a standard nucleic acid is detectable, the following oligo DNAs were used.

TABLE 7

| | | |
|---|---|---|
| Reference nucleic acid | 11-mer oligo DNA derived from | GGTGGCGTAGG |
| | part of Exon 2 of wild-type | (SEQ ID NO: 1) |
| | KRAS gene sense strand | |
| BNA consisting of sequence | 11-mer oligo DNA of antisense | CCTACGCCACC |
| complementary to reference | strand consisting of sequence | (SEQ ID NO: 4) |
| nucleic acid (may also be | complementary to the sense | |
| referred to as BNA | strand oligo DNA. However, | |
| complementary strand) | partly constituted of BNA being | |
| | unnatural nucleotide. | |
| Nucleic acid having mutation in | 11-mer oligo DNA having | GGTGACGTAGG |
| reference nucleic acid (may also | mutation at one base with | (SEQ ID NO: 3) |
| be referred to as mutation | respect to the sense strand DNA | |
| strand) | oligomer | |

In a base sequence of each DNA oligomer in the table, a mismatch site is indicated with a bold letter. Further, the nucleotide containing the base indicated by the underline is BNA having the following structure:

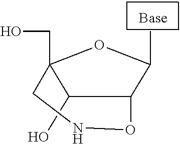

### 3. Preparation of Reaction Mixture

The following reaction mixtures 1 to 4 were prepared.

TABLE 8

| | |
|---|---|
| Components | Content |

Reaction mixture 1

| | | |
|---|---|---|
| 500 μM of reference nucleic acid aqueous solution | 2.0 | μL |
| 500 μM of BNA complementary strand aqueous solution | 2.0 | μL |
| 25 μM of fluorescent dye solution | 1.0 | μL |
| (manufactured by Biotium, trade name: EvaGreen) | | |
| Nuclease-free water | 15.0 | μL |

Reaction mixture 2

| | | |
|---|---|---|
| 500 μM of reference nucleic acid aqueous solution | 2.0 | μL |
| 500 μM of BNA complementary strand aqueous solution | 2.0 | μL |
| 25 μM of fluorescent dye solution | 1.0 | μL |
| (manufactured by Biotium, trade name: EvaGreen) | | |
| 10.0% by mass of polymer aqueous solution | 2.0 | μL |
| Nuclease-free water | 13.0 | μL |

Reaction mixture 3

| | | |
|---|---|---|
| 500 μM of mutation strand aqueous solution | 2.0 | μL |
| 500 μM of BNA complementary strand aqueous solution | 2.0 | μL |
| 25 μM of fluorescent dye solution | 1.0 | μL |
| (manufactured by Biotium, trade name: EvaGreen) | | |
| Nuclease-free water | 15.0 | μL |

Reaction mixture 4

| | | |
|---|---|---|
| 500 μM of mutation strand aqueous solution | 2.0 | μL |
| 500 μM of BNA complementary strand aqueous solution | 2.0 | μL |
| 25 μM of fluorescent dye solution | 1.0 | μL |
| (manufactured by Biotium, trade name: EvaGreen) | | |
| 10.0% by mass of polymer aqueous solution | 2.0 | μL |
| Nuclease-free water | 13.0 | μL |

Concentration of the polymer (agent for raising a Tm of a double-stranded nucleic acid) in reaction mixtures 2 and 4 was 1.0% by mass.

### 4. Creation of Melting Curve of Double Strand

Each of the prepared reaction mixtures was set in StepOnePlus Real-Time PCR System (manufactured by Thermo Fisher Scientific), heated at 95° C. for 15 seconds, and then kept at 10° C. for 1 minute. Next, while performing fluorescence measurement every 0.3 seconds, the temperature was raised to 95° C. to obtain a melting curve of a double-strand, and then held at 95° C. for 15 seconds.

### [Test Results]

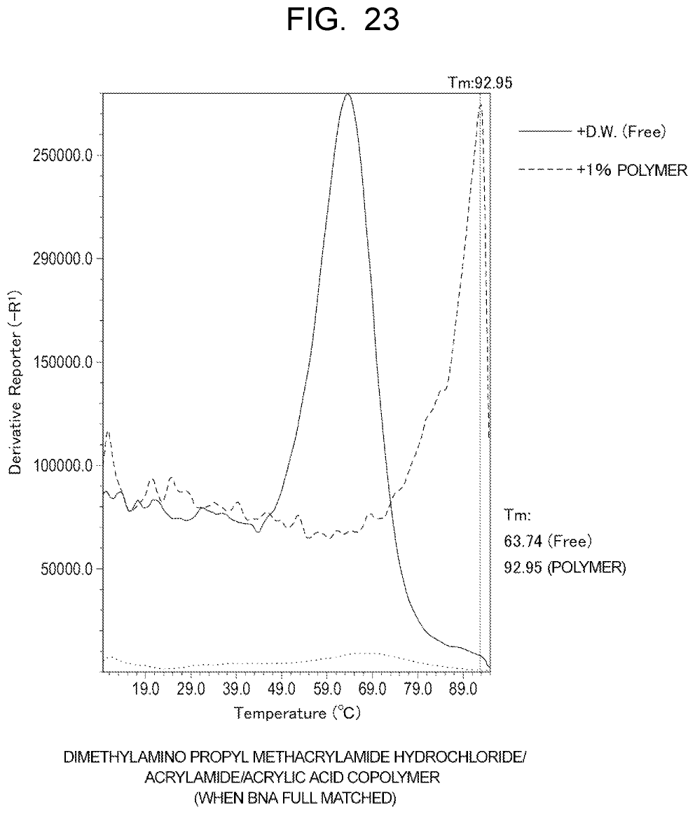

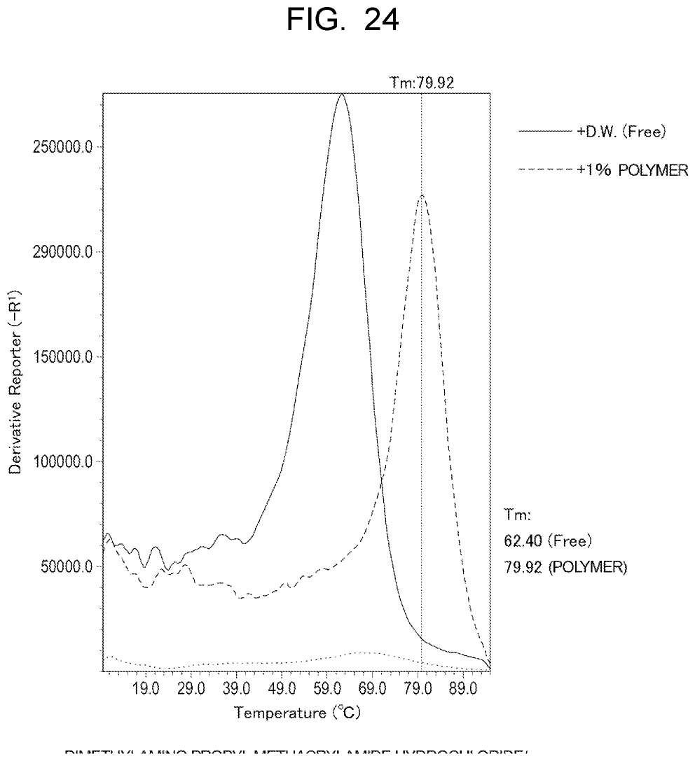

FIG. 23 shows melting curves of a double strand obtained from the reaction mixtures 1 and 2, and FIG. 24 shows melting curves of a double strand obtained from the reaction mixtures 3 and 4. As shown in FIG. 23, a Tm value of a double strand between the standard nucleic acid and the BNA complementary strand was 63.74° C. in the absence of the polymer (agent for raising a Tm of a double-stranded nucleic acid). On the other hand, a Tm value of a double strand between the standard nucleic acid and the BNA complementary strand was 92.95° C. in the presence of the polymer (agent for raising a Tm of a double-stranded nucleic acid), and a 29.21° C.-rise in the Tm value was observed.

Further, as shown in FIG. 24, a Tm value of a double strand between the mutation strand and the BNA complementary strand was 62.40° C. in the absence of the polymer (agent for raising a Tm of a double-stranded nucleic acid). On the other hand, a Tm value of a double strand between the mutation strand and the BNA complementary strand was 79.92° C. in the presence of the polymer (agent for raising a Tm of a double-stranded nucleic acid), and a 17.52° C.-rise in the Tm value was observed.

As a result, a difference of 11.69° C. was observed between the rise in the Tm value of a double strand between the standard nucleic acid and the BNA complementary strand and the rise in the Tm value of a double strand between the mutation strand and the BNA complementary strand in the presence of the polymer (agent for raising Tm of a double-stranded nucleic acid).

### Example 6

A melting curve of a double strand was obtained from each reaction mixture in a similar manner to Example 5 except that a polymer aqueous solution containing 10.0% by mass of the diallylamine hydrochloride/maleic acid copolymer that was used in Example 2, and having a pH of 7.0 was used as the agent for raising a Tm of a double-stranded nucleic acid, and then the Tm rising range (standard ΔTm) between the standard nucleic acid and the BNA complementary strand and Tm rising range (sample ΔTm) between the mutation strand and the BNA complementary strand due to the presence of a polymer (agent for raising a Tm of a double-stranded nucleic acid) were determined, and the difference (ΔΔTm) between them was calculated.

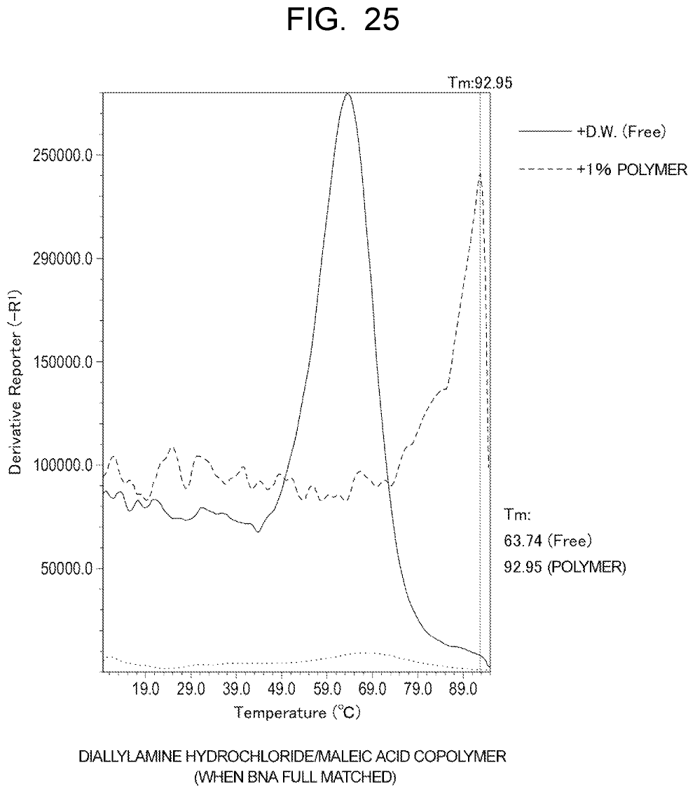

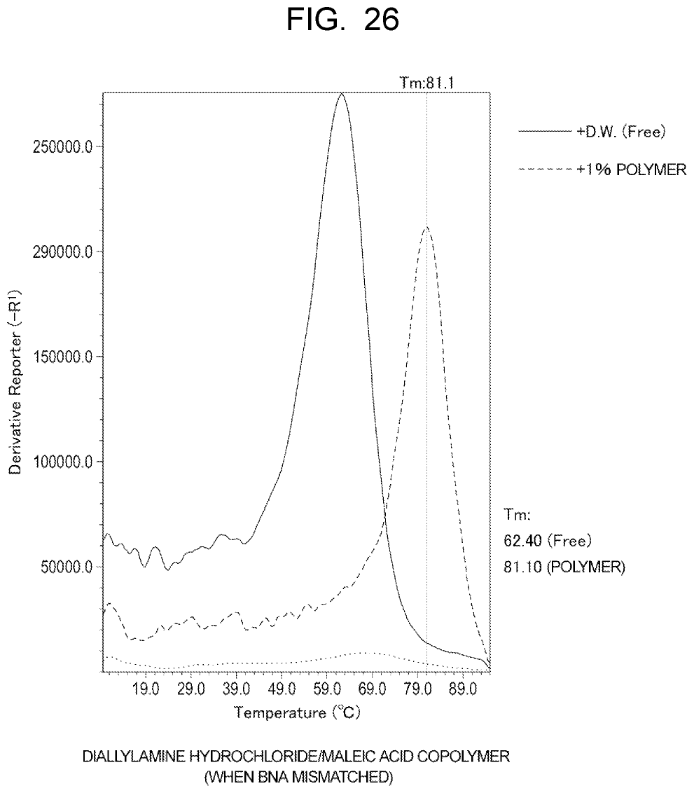

FIG. 25 shows melting curves of a double strand obtained from the reaction mixtures 1 and 2, and FIG. 26 shows melting curves of a double strand obtained from the reaction mixtures 3 and 4, respectively. As shown in FIG. 25, a Tm value of a double strand between the standard nucleic acid and the BNA complementary strand was 63.74° C. in the absence of the polymer (agent for raising a Tm of a double-stranded nucleic acid). On the other hand, a Tm value of a double strand between the standard nucleic acid and the BNA complementary strand was 92.95° C. in the presence of the polymer (agent for raising a Tm of a double-stranded nucleic acid), and a 29.21° C.-rise in the Tm value was observed.

Further, as shown in FIG. 26, a Tm value of a double strand between the mutation strand and the BNA complementary strand was 62.40° C. in the absence of the polymer (agent for raising a Tm of a double-stranded nucleic acid). On the other hand, a Tm value of a double strand between the mutation strand and the BNA complementary strand was 81.10° C. in the presence of the polymer (agent for raising a Tm of a double-stranded nucleic acid), and an 18.60° C.-rise in the Tm value was observed.

As a result, a difference of 10.61° C. was observed between the rise in the Tm value of a double strand between the standard nucleic acid and the BNA complementary strand and the rise in the Tm value of a double strand between the mutation strand and the BNA complementary strand in the presence of the polymer (agent for raising Tm of a double-stranded nucleic acid).

The test results of Examples 5 and 6 are summarized below.

TABLE 9

| | Example 5 | Example 6 |
|---|---|---|
| | Dimethylamino propyl | Diallylamine |
| Agent for raising | methacrylamide | hydrochloride/ |
| Tm of double-stranded | hydrochloride/acrylamide/ | maleic acid |
| nucleic acid | acrylic acid copolymer | copolymer |
| Molar ratio of | | |
| constituent unit | | |
| Cationic unit | 1 | 1 |
| Anionic unit | 2 | 1 |
| Nonionic unit | 1 | 0 |
| Cation/anion | 0.5 | 1 |
| BNA complementary strand | ◯ | ◯ |
| Reference ΔTm (° C.) | 29.21 | 29.21 |
| Sample ΔTm (° C.) | 17.52 | 18.60 |
| Δ ΔTm (° C.) | 11.69 | 10.61 |

1. An agent for raising a melting temperature of a double-stranded nucleic acid, comprising
an amphoteric copolymer, wherein the amphoteric copolymer comprises:
at least one type of cationic constituent unit (1) selected from the group consisting of
a constituent unit (1-1) having a structure represented by general formula (I-a) or general formula (I-b), or a structure of an acid addition salt thereof: 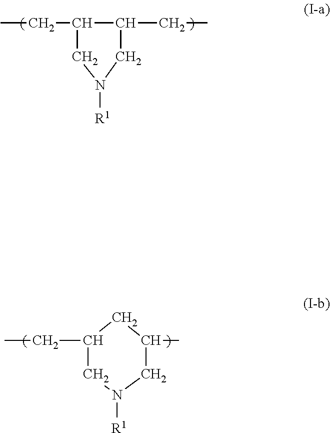
wherein R¹ represents a hydrogen atom, an alkyl group having 1 to 10 carbon atoms that may have a hydroxyl group, a cycloalkyl group having 5 to 10 carbon atoms, or an aralkyl group having 7 to 10 carbon atoms,
a constituent unit (1-4) having a structure represented by general formula (1-f), or a structure of an acid addition salt thereof: 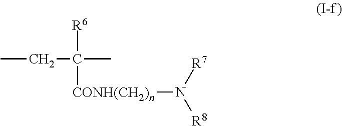
wherein R⁶ represents a hydrogen atom or a methyl group, R⁷ and R⁸ each independently represent an alkyl group having 1 to 4 carbon atoms, and n is an integer of 2 to 4; and
at least one type of anionic constituent unit (2) selected from the group consisting of
a constituent unit (2-1) having a structure represented by general formula (II-a): 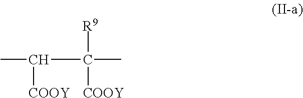
wherein R⁹ is a hydrogen or a methyl group, Y represents a hydrogen, Na, K, NH₄, ½Ca, ½Mg, ½Fe, ⅓Al, or ⅓Fe independently for each carboxy group to be bonded,
a constituent unit (2-4) having a structure represented by general formula (II-d): 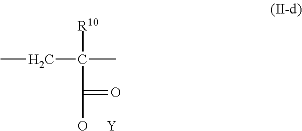
wherein R¹⁰ is a hydrogen or a methyl group, Y represents a hydrogen, Na, K, NH₄, ½Ca, ½Mg, ½Fe, ⅓Al, or ⅓Fe independently for each carboxy group to be bonded.

2. The agent for raising a melting temperature of a double-stranded nucleic acid according to claim 1, wherein the amphoteric copolymer further comprises a nonionic constituent unit (3).

3. The agent for raising a melting temperature of a double-stranded nucleic acid according to claim 2, wherein the nonionic constituent unit (3) is a constituent unit derived from a methacrylamide-based monomer, or an acrylamide-based monomer.

4. A method for detecting nucleic acid mismatching between a sample nucleic acid and a probe nucleic acid, comprising the steps of:
(1) measuring melting temperatures of a double-stranded nucleic acid between the probe nucleic acid and a nucleic acid having a base sequence complementary to the probe nucleic acid with and without adding the agent for raising a melting temperature of a double-stranded nucleic acid according to claim 1, and determining a rise value between the melting temperatures of the probe nucleic acid and the nucleic acid having a base sequence complementary to the probe nucleic acid due to the presence of the amphoteric copolymer (standard ΔTm);
(2) measuring melting temperatures of a double-stranded nucleic acid between the sample nucleic acid and the probe nucleic acid with and without adding the agent for raising a melting temperature, and determining a rise value between the melting temperatures of the double-stranded nucleic acid of the sample nucleic acid and the probe nucleic acid due to the presence of the amphoteric copolymer (sample ΔTm); and
(3) determining the presence or absence of nucleic acid mismatching based on a value (ΔΔTm) obtained by subtracting the sample ΔTm from the standard ΔTm.

5. The method for detecting nucleic acid mismatching according to claim 4, wherein it is determined that the nucleic acid mismatching is present when the ΔΔTm is 5.0° C. or more.

6. A kit for detecting nucleic acid mismatching between a sample nucleic acid and a probe nucleic acid, comprising:
the probe nucleic acid;
a nucleic acid having a base sequence complementary to the probe nucleic acid; and
the agent for raising a melting temperature according to claim 1.

7. A method for detecting mutation with respect to a standard nucleic acid in a sample nucleic acid, comprising the steps of:
(1) measuring melting temperatures of a double-stranded nucleic acid between the standard nucleic acid and a nucleic acid having a base sequence complementary to the standard nucleic acid with and without adding the agent for raising a melting temperature according to claim 1, and determining a rise value (standard ΔTm) between the melting temperatures of the double-stranded nucleic acid between the standard nucleic acid and the nucleic acid having the base sequence complementary to the standard nucleic acid due to the presence of the amphoteric copolymer;
(2) measuring melting temperatures of a double-stranded nucleic acid of the sample nucleic acid and the nucleic acid having a base sequence complementary to the standard nucleic acid with and without adding the agent for raising a melting temperature, and determining a rise value (sample ΔTm) between the melting temperatures of the double-stranded nucleic acid between the sample nucleic acid and the nucleic acid having the base sequence complementary to the standard nucleic acid due to the presence of the amphoteric copolymer; and
(3) determining the presence or absence of mutation based on a value (ΔΔTm) obtained by subtracting the sample ΔTm from the standard ΔTm.

8. The method for detecting mutation according to claim 7, wherein it is determined that mutation is present when the ΔΔTm is 5.0° C. or more.

9. A kit for detecting mutation between a sample nucleic acid and a standard nucleic acid, comprising:
the standard nucleic acid;
a nucleic acid having a base sequence complementary to the standard nucleic acid; and
the agent for raising a melting temperature according to claim 1.

10. A method for raising a melting temperature of a double-stranded nucleic acid, comprising mixing the agent for raising a melting temperature of a double-stranded nucleic acid according to claim 1 with the double-stranded nucleic acid in a solution.

11. The method according to claim 10, wherein the amphoteric copolymer further comprises a nonionic constituent unit (3).

12. The method according to claim 10, wherein the nonionic constituent unit (3) is a constituent unit derived from a methacrylamide-based monomer, or an acrylamide-based monomer.
